(19) United States
(12) Patent Application Publication
(10) Pub. No.: US 2002/0068301 A1
(43) Pub. Date: Jun. 6, 2002

(54) CYCLIC PEPTIDE LIBRARIES AND METHODS OF USE THEREOF TO IDENTIFY BINDING MOTIFS

(76) Inventors: HUNG-SEN LAI, BOSTON, MA (US); MICHAEL B. YAFFE, SOMERVILLE, MA (US); ZHOU SONGYANG, BROOKLINE, MA (US); KERMIT L. CARRAWAY III, WATERTOWN, MA (US); LEWIS C. CANTLEY, CAMBRIDGE, MA (US)

Correspondence Address: TESTA, HURWITZ & THIBEAULT, LLP, HIGH STREET TOWER, 125 HIGH STREET, BOSTON, MA, 02110, US

(21) Appl. No.: 08/864,392

(22) Filed: May 28, 1997

(51) Int. Cl. G01N 33/53; C12N 9/12

(52) U.S. Cl. 435/7.1; 435/194

(57) ABSTRACT

Methods for determining an optimal binding motif for a binding compound are provided in which the binding compound is contacted with an oriented degenerate cyclic peptide library (ODCPL) under conditions which allow for interaction between the binding compound and the ODCPL such that a complex is formed between the binding compound and a subpopulation of library members capable of interacting with the binding compound. The subpopulation of library members capable of interacting with the binding compound is then separated from library members that are incapable of interacting with the binding compound. The subpopulation of library members capable of interacting with the binding compound is linearized to form a subpopulation of linearized library members. The amino acid sequence of the subpopulation of linearized library members is determined and an amino acid sequence motif is then determined for an interaction site of the binding compound, based upon the relative abundance of different amino acid residues at each degenerate position within the linearized library members. Oriented degenerate cyclic peptide libraries, and methods for purifying cyclic peptides from linear peptides, are also provided.

### BACKGROUND OF THE INVENTION

Many biological responses are mediated by the interaction of a binding protein with a target compound. Examples of such binding interactions include the interaction of an enzyme with its substrate (e.g., the interaction of kinases, proteases, phosphatases etc. with their substrates), the interaction of antibodies with antigens, the interaction of receptors with ligands and the interaction of SH2 domains with phosphotyrosine-containing targets. The specificity of a particular binding protein, such as an enzyme, typically has been determined by identifying a number of natural substrates for the protein, obtaining the sequence of these substrates and then comparing the sequences of these substrates to define a consensus motif for substrate binding.

For example, information on the specificity of protein kinases has typically come from locating the phosphorylation sites on in vivo and/or in vitro substrates of the kinase and determining a consensus motif for the phosphorylation sites by comparing the sequences of the substrates. (See e.g., Taylor et al., (1990) Ann. Rev. Biochem. 59:971-1005; Cheng, et al. (1991) J. Biol. Chem. 266:17919-17925; Walsh et al. (1990) in Peptides and Protein Phosphorylation. (B.E. Kemp, ed.) CRC Press Inc., pp. 43-84; Gaehlen and Harrison (1990) in Peptides and Protein Phosphorylation. (B.E. Kemp, ed.) CRC Press Inc., pp. 239-254). These types of studies have demonstrated the importance of the primary amino acid sequence around the site of phosphorylation in determining the in vivo specificity of protein kinases. Once a binding motif has been defined in this manner, synthetic peptides have been constructed based upon the consensus sequence motif and individual amino acids have been replaced one by one in order to determine the importance of particular amino acids on the K_{M} or Vₘₐₓ of the phosphorylation reaction.

There are severe limitations, however, to this approach for determining the specificity of a binding protein. The procedure is quite expensive and laborious since each amino acid residue within a putative binding motif must be altered individually to evaluate its importance. Moreover, this approach does not necessarily identify all the residues critical for substrate specificity. Furthermore, an optimal substrate sequence is not likely to be determined unless each residue is changed to every other possible amino acid residue individually and then evaluated. For example, based upon an estimation of 9 to 12 amino acid residues of a substrate contacting the active site cleft of a kinase there would be approximately 1.024×10¹³(20¹⁰) distinct peptides to consider. Moreover, in many cases this approach is not feasible because in vivo substrates for the binding protein cannot be determined with certainty. For example, if an extracellular signal activates multiple kinases which phosphorylate multiple substrates or if a signal activates a cascade of kinases it is difficult to determine which kinase phosphorylates which substrate. An even more difficult problem in determining the substrate specificity of a particular binding protein, especially enzymes, is that their critical in vivo substrates are often proteins which are present in vivo in very low abundance and thus are not easily detectable by in vivo assays.

Thus, alternative methods to that of isolating and examining the sequences of native substrates are desirable for determining the substrate specificity of a binding protein. Songyang et al. (Cell(1993)72:767-778) describe a method for determining the sequence specificity of the peptide-binding sites of SH2 domains using oriented degenerate phosphopeptide libraries. In this approach a library of peptides containing a fixed phosphotyrosine residue is used to select the optimal phosphopeptide substrates for a particular SH2 domain. A similar approach has been applied to the determination of optimal substrates for protein kinases (see U.S. Pat. No. 5,532,167). These methodologies utilized linear peptide libraries composed entirely of naturally-occurring α-amino acids. One drawback to use of such linear, natural peptides is that they may not appropriately mimic the conformation of a target binding site within a larger protein substrate, due to the high flexibility of linear peptides in solution. Moreover, linear peptides composed entirely of natural amino acids are highly susceptible to degradation by proteases in vivo, which could limit the ability to use such peptides as pharmacological agents. Accordingly, improved properties for identifying binding motifs for binding compounds are needed.

### SUMMARY OF THE INVENTION

This invention provides improved methods and compositions for identifying binding motifs for binding compounds, which utilize cyclic peptides composed of natural and/or unnatural α-amino acids. The methods and compositions of the invention allow for determination of an optimal binding motif for a binding compound within the context of a cyclic peptide. Cyclic peptides comprising these optimal motifs then can be used as pharmacological agents to modulate the biological activity of the binding compound. The advantages of the methods and compositions of the invention include that since the cyclic peptides exhibit constrained flexibility compared to linear forms, the cyclic peptides are more likely to mimic the conformation of a target binding site within a larger protein substrate. Furthermore, cyclic peptides are more resistant to degradation compared to linear forms and thus are more amenable to pharmaceutical use.

The methods of the invention for identifying binding motifs for binding compounds can be applied to essentially any compound that has affinity for cyclic peptides. Non-limiting examples of binding compounds that can be analyzed according to the methods of the invention include kinases (e.g., protein serine/threonine kinases, protein tyrosine kinases, lipid kinases), phosphatases (e.g., protein phosphatases, lipid phosphatases), proteases (e.g., serine proteases, cysteine proteases), SH2 domains, SH3 domains, antibodies, WW domains, PTB domains, PDZ domains, LIM domains, pleckstrin homology domains, zinc finger domains, extracellular growth factors and receptors, adhesion molecules, intercellular signalling molecules, 7-transmembrane receptor proteins, ion channels, methyltransferases, ubiquitinating enzymes and peptidyl-transferases.

One aspect of the invention pertains to a method for determining an amino acid sequence motif for an interaction site of a binding compound. The method involves contacting an oriented degenerate cyclic peptide library (ODCPL) with a binding compound under conditions which allow for interaction between the binding compound and the ODCPL. The ODCPL includes library members having an identifiable amino acid residue at a fixed non-degenerate position. The binding compound interacts with the ODCPL such that a complex is formed between the binding compound and a subpopulation of library members capable of interacting with the binding compound. The subpopulation of library members capable of interacting with the binding compound is then separated from library members that are incapable of interacting with the binding compound. The subpopulation of library members capable of interacting with the binding compound is linearized to form a subpopulation of linearized library members. The amino acid sequence of the subpopulation of linearized library members is determined. An amino acid sequence motif is then determined for an interaction site of the binding compound, based upon the relative abundance of different amino acid residues at each degenerate position within the linearized library members.

Another aspect of the invention pertains to oriented degenerate cyclic peptide libraries (ODCPL). In a preferred embodiment, the ODCPL comprises cyclic peptides having the formula:

[chemical structure]

Zₐₐ is a non-degenerate natural or unnatural α-amino acid. Xₐₐ is any natural or unnatural α-amino acid. R—R′ is a dipeptide specifically cleavable under cleavage conditions to allow for linearization of the peptide. n and m are each independently selected from 0-10 inclusive, with the proviso that if n is 0, m is selected from 1-10 inclusive and if m is 0, n is selected from 1-10 inclusive.

The invention still further provides a method for purifying cyclic peptides from linear peptides. The method involves first providing a mixture of cyclic peptides and linear peptides. The mixture is then contacted with a blocking agent that reacts with the free amino termini of the linear peptides to form amino-protected linear peptides. The mixture then is contacted with a binding agent that is capable of interacting with the amino-protected linear peptides but incapable of interacting with the cyclic peptides. Finally, the amino-protected linear peptides are separated from the cyclic peptides to thereby purify the cyclic peptides. In a preferred embodiment, the blocking agent is a biotin, which biotinylates the free amino-termini of the linear peptides, and the binding agent is a biotin-binding agent, such as avidin or streptavidin. For example, biotinylated linear peptides can be separated from cyclic peptides by passing the mixture over an avidin column.

The invention also provides a method for determining an amino acid sequence motif for an interaction site of a protease. The method includes contacting an oriented degenerate cyclic peptide library (ODCPL) with a protease under conditions which allow for interaction between the protease and the ODCPL, wherein the ODCPL includes library members having an identifiable amino acid residue at a fixed non-degenerate position. The protease interacts with the ODCPL such that a complex is formed between the protease and a subpopulation of library members capable of interacting with the protease. The subpopulation of library members capable of interacting with the protease to form a subpopulation of linearized library members is then linearized. The amino acid sequence of the subpopulation of linearized library members is determined and an amino acid sequence motif for an interaction site of the protease, based upon relative abundance of different amino acid residues at each degenerate position within the linearized library members is also determined. An example of a suitable protease is chymotrypsin.

### DESCRIPTION OF THE INVENTION

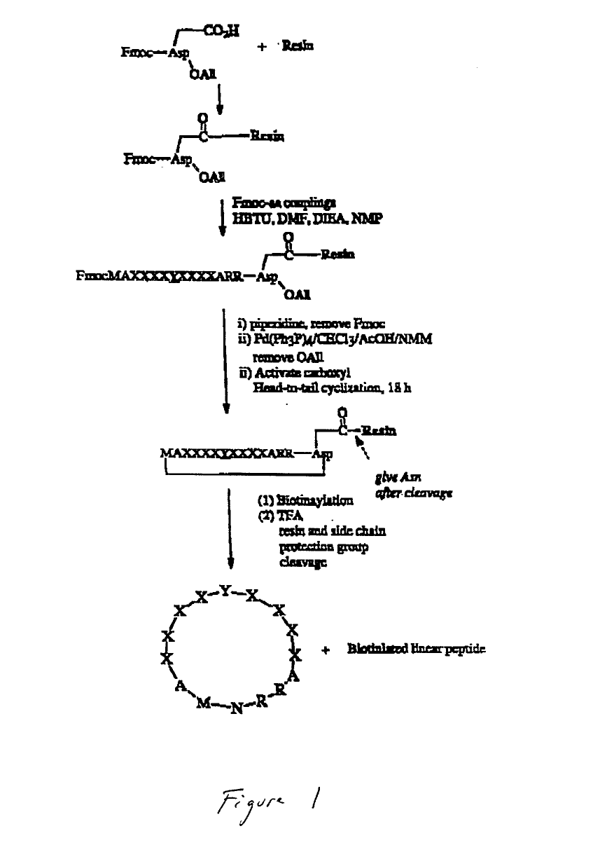

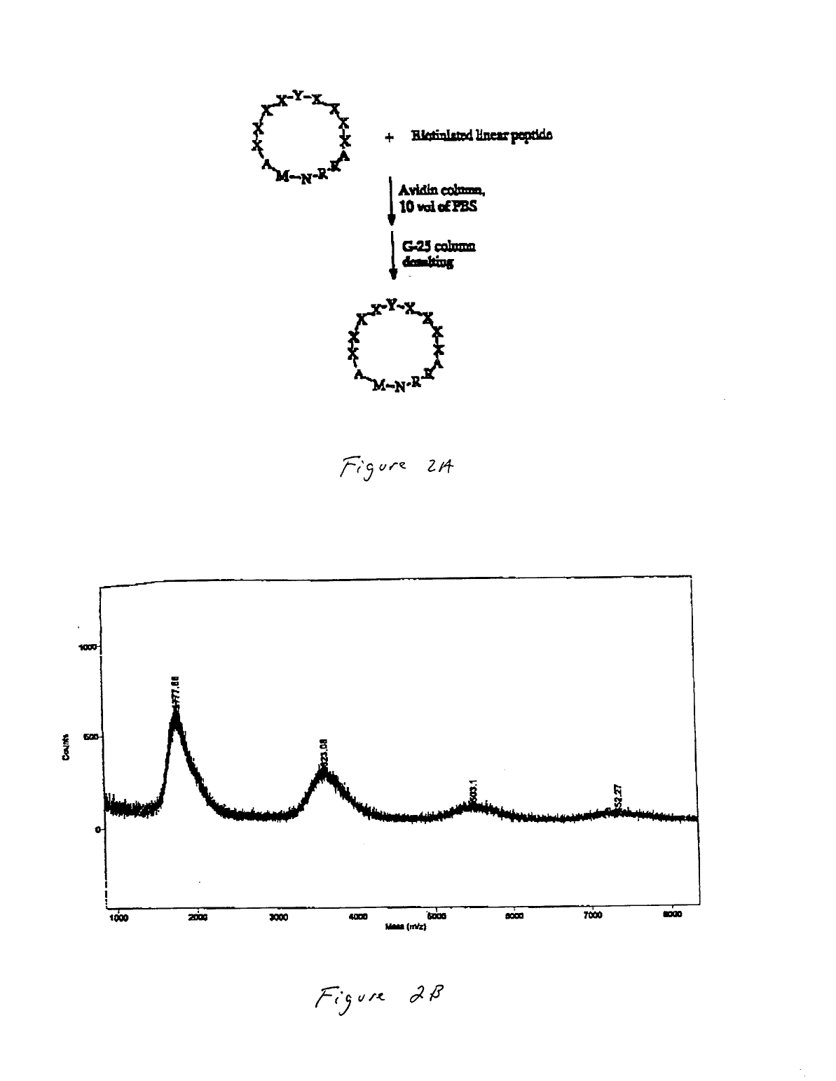

FIG. 1 is a schematic diagram of on-resin cyclic peptide synthesis using alpha-allyl esters.

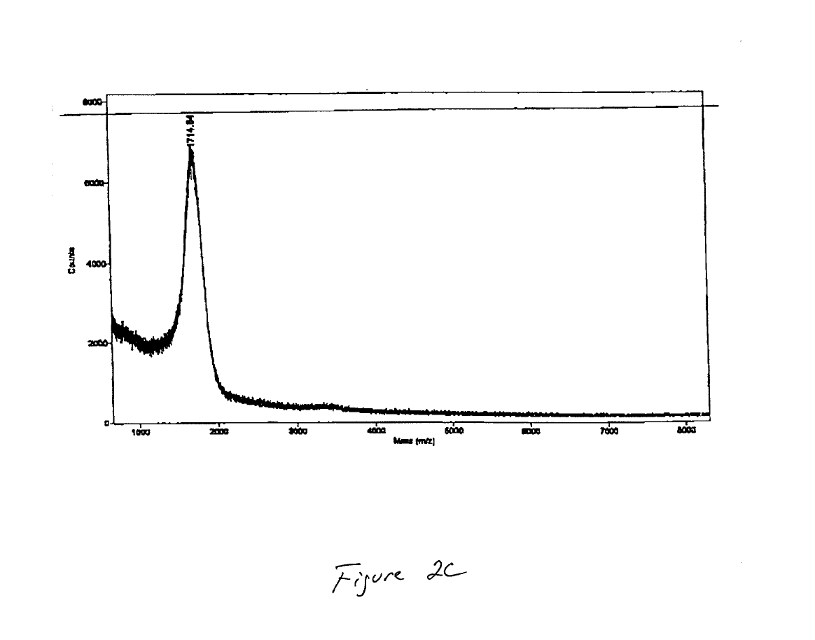

FIG. 2A is an exemplary schematic diagram of separating cyclic peptides from linear peptides.

FIG. 2B shows a mass spectrum of a mixture of peptides.

FIG. 2C shows the mass spectrum of 2B after removal of contaminants.

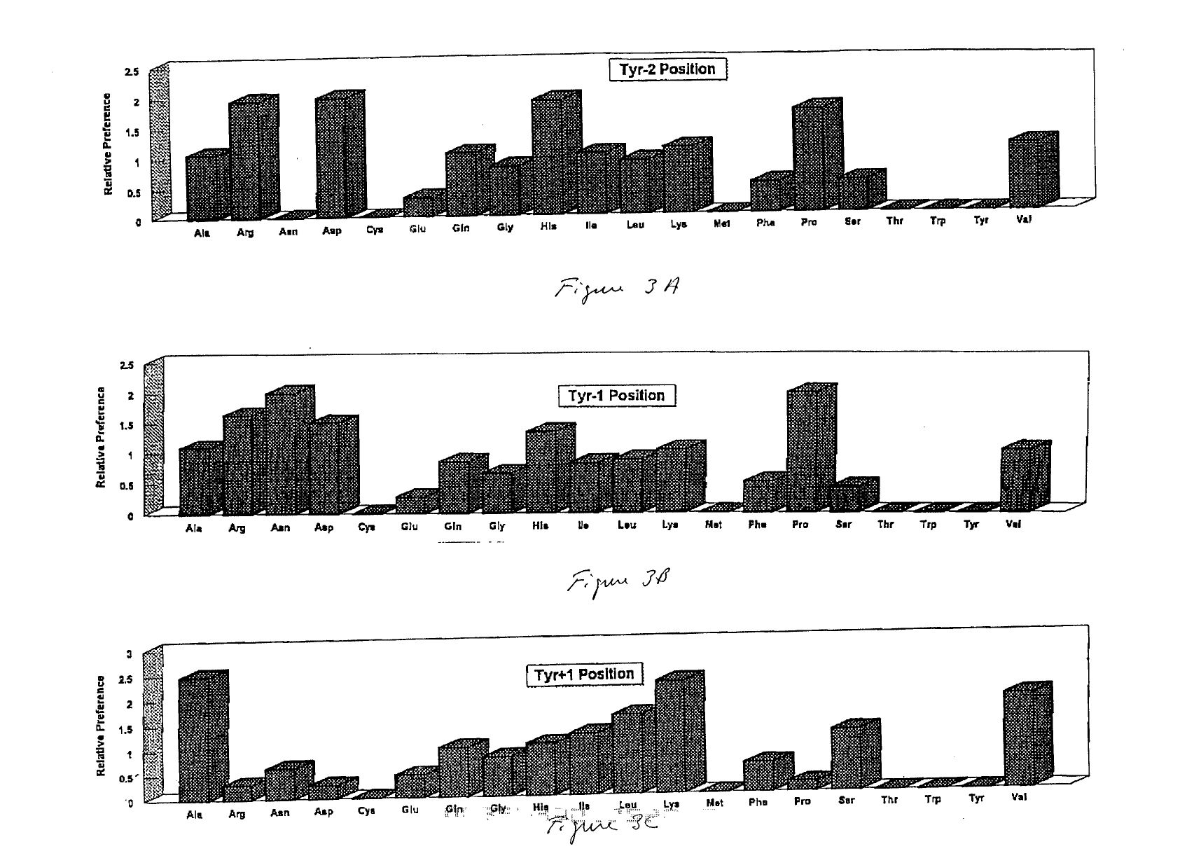

FIGS. 3A, 3B and 3C are bar graphs depicting the relative abundance of each amino acid residue at degenerate positions Tyr−2, Tyr−1 and Tyr+1, respectively, of peptides from members of an oriented degenerate cyclic peptide library that were cleaved by chymotrypsin.

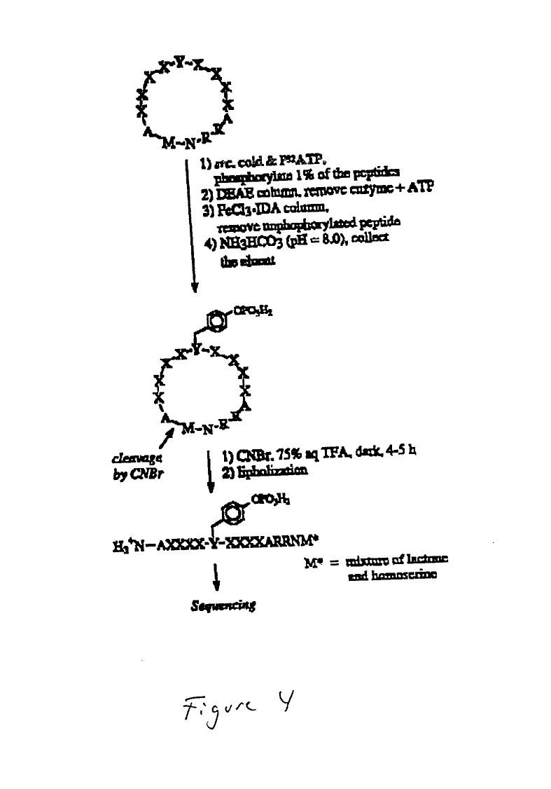

FIG. 4 is a schematic which depicts the cleavage of a phosphorylated oriented degenerate cyclic peptide.

### DETAILED DESCRIPTION OF THE INVENTION

This invention pertains to methods for determining an amino acid sequence motif for an interaction site of a binding compound, oriented degenerate cyclic peptide libraries (ODCPL) for use in such methods, and methods for purifying cyclic peptides from linear peptides. The invention allows for the determination of the optimal interaction site motif for a particular binding compound within the context of a cyclic peptide. This method has the advantage that it can be used to determine an interactive site for any binding compound, regardless of whether native substrates for that binding compound have been identified. Furthermore, since the method involves selection of oriented degenerate cyclic peptides which interact most readily with a binding compound, the amino acid sequence motif that is determined represents the optimal interactive site for that binding compound.

Based upon the amino acid sequence motif of the interaction site of a binding compound determined by the method of the invention, oriented degenerate cyclic peptides and peptide libraries can be made which are substrates for that particular binding compound. Cyclic peptides comprising the most preferred amino acid residues of a sequence motif represent optimal cyclic substrates for the binding compound. Cyclic peptides, or modified forms thereof, designed based upon the interaction site motifs provided by the invention can be used to detect and quantitate binding compounds. Furthermore, due to their high affinity for the binding compound, such cyclic peptides can be used to modulate the activity of the binding compound. Pseudosubstrates and analogs can also be designed based upon the amino acid sequence motifs provided by the invention. In a preferred embodiment, the amino acid sequence motif for the optimal interaction site is used to design pharmaceutical agents for modulating the biological activity of the binding compound.

So that the invention may be more readily understood, certain terms are first defined.

The term “cyclic peptide” is intended to include peptides composed of natural and/or unnatural α-amino acids that have been cyclized by formation of an intrapeptide bond. This intrapeptide bond preferably is formed between the amino-terminal amino group of the peptide and the carboxy-terminal carboxy group of the peptide (referred to as “head to tail” cyclization), although the term “cyclic peptide” is also intended to include peptides that have been cyclized by intrapeptide bond formation involving a side chain group(s). The term “unnatural α-amino acids” is intended to include analogs, derivatives and congeners of naturally occurring amino acids. For example, unnatural amino acids can have lengthened or shortened side chains or variant side chains with appropriate functional groups. Also included are the D and L stereoisomers of an amino acid when the structure of the amino acid admits of stereoisomeric forms.

The term “oriented degenerate cyclic peptide library (ODCPL)” is intended to include populations of cyclic peptides (composed of natural and/or unnatural α-amino acids) in which different amino acid residues are present at the same position in different peptides within the library. For example, a population of peptides of 10 amino acids in length in which the amino acid residue at position 5 of the peptides can be any one of the twenty natural amino acids would be a degenerate peptide library. A position within the peptides which is occupied by different amino acids in different peptides is referred to herein as a “degenerate position”, whereas a position within the peptides which is occupied by the same amino acid in different peptides is referred to herein as a “non-degenerate position”. The “oriented degenerate cyclic peptide library” used in the method of the invention is composed of cyclic peptides which have at least one amino acid residue at a fixed, non-degenerate position. The term “fixed position” indicates that the peptides contained within the library all have the same non-degenerate amino acid residue at that particular position within the peptides.

The term “binding compound” is intended to include all compounds which can interact with an interaction site on a cyclic peptide by one or more mechanisms. Examples of suitable binding compounds include kinases (e.g., protein serine/threonine kinases, protein tyrosine kinases, lipid kinases), phosphatases (e.g., protein phosphatases, lipid phosphatases), proteases (e.g., serine proteases, cysteine proteases), SH2 domains, SH3 domains, antibodies, WW domains, PTB domains, PDZ domains, LIM domains, pleckstrin homology domains, zinc finger domains, extracellular growth factors and receptors, adhesion molecules, intercellular signalling molecules, 7-transmembrane receptor proteins, ion channels, methyltransferases, ubiquitinating enzymes and peptidyl-transferases.

The term “interaction” is intended to include those attractive forces which physically combine a binding compound with an interaction site on an oriented degenerate cyclic peptide. Such attractive forces include hydrophobic interactions, hydrophilic interactions, covalent binding, ionic binding, charged interactions, etc.

The phrase “an amino acid sequence motif for an interaction site” is intended to describe a composite amino acid sequence which represent a consensus sequence for an interaction site recognized by a binding compound. An amino acid sequence motif for an interaction site typically encompasses the region including and surrounding an amino acid residue(s) which specifically and preferentially interacts with a binding compound.

Various aspects of the invention are described in further detail below:

I. Methods for Identifying Interaction Site Motifs for Binding Compounds

The invention provides a method for determining an amino acid sequence motif for an interaction site of a binding compound. The method of the invention involves:

contacting an oriented degenerate cyclic peptide library (ODCPL) with a binding compound under conditions which allow for interaction between the binding compound and the ODCPL, wherein the ODCPL comprises library members having an identifiable amino acid residue at a fixed non-degenerate position;

allowing the binding compound to interact with the ODCPL such that a complex is formed between the binding compound and a subpopulation of library members capable of interacting with the binding compound;

separating the subpopulation of library members capable of interacting with the binding compound from library members that are incapable of interacting with the binding compound;

linearizing the subpopulation of library members capable of interacting with the binding compound to form a subpopulation of linearized library members;

determining the amino acid sequence of the subpopulation of linearized library members; and

determining an amino acid sequence motif for an interaction site of the binding compound, based upon relative abundance of different amino acid residues at each degenerate position within the linearized library members.

The specific conditions under which the binding compound and ODCPL are contacted will vary depending upon the particular binding compound and ODCPL used but are chosen such that a complex can form between the binding compound and a subpopulation of library members that are capable of interacting with the binding compound. When the binding compound is an enzyme, the enzyme and the ODCPL are contacted under conditions that maintain the enzymatic activity of the enzyme (e.g., a kinase is incubated with the ODCPL under conditions that allow for phosphorylation of the library members by the kinase).

After complexes have formed between the binding compound and the subpopulation of library members that are capable of interacting with the binding compound (referred to as the “binding subpopulation”), the binding subpopulation is separated from the non-binding subpopulation (e.g., those library members that do not interact with the binding compound) and the binding subpopulation is linearized. For most binding compounds, the separation step will precede the linearization step. However, in certain embodiments, the linearization step may be performed before the separation step.

The method for separating the binding subpopulation of library members from the non-binding subpopulation of library members will depend upon the particular binding compound used. For example, in one embodiment, the binding compound is immobilized on a solid support (e.g., a column) and the binding subpopulation of library members remains bound to the immobilized binding compound while the non-binding subpopulation is washed away. Standard methods for affinity chromatography can be used to afford such separation. Binding compounds can be immobilized to a solid support using methods known in the art. For example, the binding compound can be prepared as a glutathione-S-transferase (GST) fusion protein and immobilized by binding the GST fusion protein to glutathione agarose beads. Such an approach is suitable for many types of binding compounds but is particularly preferred for binding domains that mediate protein-protein interactions (such as SH2 and SH3 domains) but that do not have enzymatic activity.

In another embodiment, in which the binding compound has an enzymatic activity, separation of the binding subpopulation of library from the non-binding subpopulation can be based on enzymatic modification of the binding subpopulation by the binding compound. For example, when the binding compound is a kinase, the binding subpopulation of library members becomes phosphorylated while the non-binding subpopulation remains nonphosphorylated (discussed further below). Accordingly, phosphorylated peptides can be separated from nonphosphorylated peptides to achieve separation of the binding subpopulation from the non-binding subpopulation. Similarly, when the binding compound is a phosphatase, the binding subpopulation of library members becomes dephosphorylated while the non-binding subpopulation remains phosphorylated (discussed further below). Accordingly, nonphosphorylated peptides can be separated from phosphorylated peptides to achieve separation of the binding subpopulation from the non-binding subpopulation.

The method of linearization of the binding subpopulation also will depend on the particular binding compound and ODCPL used in the method. In a preferred embodiment, the ODCPL comprises a fixed dipeptide having the amino acid sequence methionine-alanine. Specific cleavage of the library members at this dipeptide can be achieved using cyanogen bromide. In another preferred embodiment, the binding compound used in the method is a protease and the library members are linearized by cleavage mediated by the protease (discussed further below).

The linearized library members are sequenced by standard amino acid sequencing techniques (e.g., Edman degradation). Automated peptide sequencers can be used to determine the amino acid sequence of the library members. Preferably, the ODCPL used is a soluble synthetic peptide library and the linearized subpopulation of peptides is sequenced as a bulk population using an automated peptide sequencer. This approach provides information on the abundance of each amino acid residue at a given cycle in the sequence of the complexed mixture, most importantly at the degenerate positions. For each degenerate position in the selected linearized peptides (e.g., the binding subpopulation), a relative abundance value can then be calculated by dividing the abundance of a particular amino acid residue at that position after library screening (e.g., after peptide complexation and separation) by the abundance of the same amino acid residue at that position in the starting library. Thus, the relative abundance (RA) of an amino acid residue Xaa at a degenerate position in the cyclic peptide library can be defined as:

RA=amount of Xaa in the population of selected linearized peptides amount of Xaa in the original oriented degenerate cyclic peptide library

The relative abundance value may be corrected for background contamination as described further below. Amino acid residues which are neither enriched for nor selected against in the population of cyclic peptides which can serve as substrates for the binding compound will have a relative abundance of 1.0. Those amino acid residues which are preferred at a particular degenerate position (e.g., residues which are enriched at that position in the complexed peptides) will have a relative abundance greater than 1.0. Those amino acid residues which are not preferred (e.g., residues which are selected against at that position in the complexed peptides) will have a relative abundance less than 1.0. Based upon the relative abundance values for each amino acid residue at a degenerate position, preferred amino acid residues, e.g., amino acid residues with a relative abundance greater than 1.0, can be identified at that position.

Based upon the relative abundance of different amino acid residues at each degenerate position within the population of phosphorylated peptides, an amino acid sequence motif for a phosphorylation site of the protein kinase can be determined. The amino acid sequence motif encompasses the degenerate region of the peptides. The particular amino acid residues chosen for the motif at each degenerate position are those which are most abundant at each position. Thus, an amino acid residue(s) with a relative abundance value greater than 1.0 at a particular position can be chosen as the amino acid residue(s) at that position within the amino acid sequence motif. Alternatively, a higher relative abundance value can be used as the basis for inclusion of an amino acid residue to create an even more preferred phosphorylation site for a kinase. For example, an amino acid residue(s) with a relative abundance value equal to or greater than 1.5 at a particular position can be chosen as the amino acid residue(s) at that position within the amino acid sequence motif.

In a preferred embodiment, the ODCPL used in the method is a soluble synthetic library comprising cyclic peptides comprising a formula:

[chemical structure]

wherein Zₐₐ is a non-degenerate natural or unnatural α-amino acid, Xₐₐ is any natural or unnatural α-amino acid, R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive. In a preferred embodiment, R—R′ is methionine-alanine, which allows for linearization of the cyclic peptides by cleavage of any Met-X bond with cyanogen bromide.

In the following subsections, application of the method of the invention to nonlimiting examples of different types of binding compounds is described further.

A. Kinases as Binding Compounds

In one embodiment, a binding compound used in the method is a kinase. Examples of kinases include protein serine kinases, protein threonine kinases, protein tyrosine kinases and lipid kinases. When the binding compound is a kinase, the ODCPL is constructed such that is contains a phosphorylatable amino acid residue at a fixed, non-degenerate positions. As used herein, a “phosphorylatable” amino acid residue is an amino acid residue that is capable of being phosphorylate by a kinase. In a preferred embodiment, an ODCPL for use with serine, threonine or tyrosine kinases comprises cyclic peptides comprising a formula:

[chemical structure]

wherein Zₐₐ is a non-degenerate phosphorylatable amino acid selected from the group consisting of serine, threonine and tyrosine, Xₐₐ is any natural or unnatural α-amino acid, R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive.

For protein-serine/threonine specific kinases, the ODCPL preferably comprises cyclic peptides comprising a formula:

[chemical structure]

wherein Zₐₐ is ay non-degenerate phosphorylatable amino acid selected from the group consisting of serine and threonine, Xₐₐ is any natural or unnatural α-amino acid, R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive.

For protein-tyrosine specific kinases, the ODCPL preferably comprises cyclic peptides comprising a formula:

[chemical structure]

wherein Zₐₐ is tyrosine, Xₐₐ is any natural or unnatural α-amino acid, R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive.

In the foregoing ODCPLs, preferably Zaa is the only phosphorylatable amino acid within the cyclic peptides. However, it is possible for the ODCPL to contain additional phosphorylatable residues to the fixed residue and to estimate the degree of background which will occur when using such a library and take this background into consideration when evaluating the results of the library screening (discussed further below).

The ODCPL is contacted with a kinase under conditions which allow for phosphorylation of a substrate by the kinase. The kinase is allowed to phosphorylate cyclic peptides within the ODCPL having a preferred sequence for phosphorylation by the kinase, thereby forming a population of phosphorylated cyclic peptides. The phrase “contacted under conditions which allow for phosphorylation of a substrate by the kinase” is intended to include any form of combining or incubating together of the kinase and the library under conditions which enable phosphorylation of substrate proteins or peptides by the kinase. Thus, these conditions will include the presence of ATP (or an ATP analogue) as a phosphate donor molecule (or analogue thereof). The phosphate of the phosphate donor molecule can be labeled, e.g., radiolabeled, to label peptides which become phosphorylated by the kinase (e.g., to allow for their detection by detecting the radiolabel). For example, ³²P-γ-ATP can be used as the phosphate donor.

Following phosphorylation of phosphorylatable cyclic peptides within the ODCPL, the phosphorylated peptides are separated from the remaining non-phosphorylated cyclic peptides of the library. A preferred method for separating phosphorylated peptides from non-phosphorylated peptides is by binding the phosphorylated peptides to a ferric column. This type of column has been used previously to separate tryptic phosphopeptide fragments of phosphorylated proteins from non-phosphorylated tryptic fragments (Muszynska et al., (1986) Biochem. 25: 6850-6853; Muszynska et al., (1992) J. Chromatography 604:19-28). However, previously described protocols could not be used because the phosphopeptide were eluted from the column in such a way that they could not then be sequenced, which is the necessary subsequent step in the procedure (e.g., the washing and elution buffers were incompatible with subsequent sequencing of the phosphopeptides). The column washing and elution conditions were modified to allow for subsequent sequencing of the eluted phosphopeptides. In the modified procedure, the peptide mixture is loaded onto the column in high salt buffer of about pH 5.5-6.0(e.g., 50 mM MES, 1 M NaCl, pH 5.5). In this buffer, phosphorylated peptides bind to the column whereas non-phosphorylated peptides flow through the column. Next, the column is washed with a very low salt buffer of about pH 6.0 (e.g., 2 mM MES, pH 6.0) to remove contaminating non-phosphorylated peptides and excess salt from the column. Finally, the phosphopeptides are eluted from the column with a buffer of about pH 8.0(e.g., 500 mM NH₄CO₃, pH 8.0). Procedures for quantitative separation of phosphorylated peptides from non-phosphorylated peptides are described further in U.S. Pat. No. 5,532,167.

Alternatively, since many kinases are known to be capable of using ATP-γ-S as a thio-phosphate donor to phosphorylate proteins and peptides, a mercury (Hg2+) column (pChloromercuribenzoate-Agarose: Pierce) could be used for separating thio-phosphorylated peptides from non-phosphorylated peptides. Such columns have been used in the past to bind thio-phosphorylated nucleotides and peptides (Sun, I. Y-C., and Allfrey, V. G. (1982) J. Biol. Chem. 257:1347-1353 and Sun et al., (1980) J. Biol. Chem. 255:742-747). Since any thiol group will bind to the mercury column, Cys cannot be present in the library if the mercury column is used. Also, the kinase reaction is typically about 5 fold slower with ATP-γ-S so more enzyme or a longer incubation time is likely to be necessary.

The use of a mercury column to separate thio-phosphorylated peptides from non-thio-phosphorylated peptides can allow for the inclusion of phosphotyrosine, phosphoserine or phosphothreonine at degenerate positions in the library. In this case, many peptides within the library would be phosphorylated during the synthesis of the library and the kinase reaction would then be performed with ATP-γ-S as a thio-phosphate donor to thiophosphorylate substrates of the kinase. The thiophosphorylated peptides would then be separated from the non-thio-phosphorylated peptides with a mercury column.

Another theoretical alternative approach to separating phosphorylated peptides from non-phosphorylated peptides is to use an antibody-affinity column. For example, an anti-phosphotyrosine antibody-column can be used to separate peptides phosphorylated on tyrosine from non-phosphorylated peptides. However, because some anti-phosphotyrosine antibodies have some specificity for the amino acids surrounding the phosphorylated tyrosine, the antibody column could impose additional selection on the mixture of phosphorylated peptides. Thus, it is likely that some peptides which can be substrates for the kinase (e.g., a proportion of the phosphorylated peptide mixture) might be lost during the column separation step, thereby artifactually altering the mixture of peptides which is sequenced.

Prior to the step of separating the phosphorylated (or thio-phosphorylated) peptides from non-phosphorylated peptides, additional purification steps may be added to remove other components of the kinase reaction, such as the kinase itself and the free phosphate donor. For example, the kinase can be bound to a solid support, such as a bead, during the kinase reaction and then the supernatant, containing the phosphorylated and non-phosphorylated peptides can be removed, thereby separating the kinase from the peptide. Chromatography can be used to separate the free phosphate donor (e. g, ATP) from the peptides, for example a DEAE column can be used.

Following separation of the phosphorylated cyclic peptides from nonphosphorylated cyclic peptides, the phosphorylated cyclic peptides are linearized and subjected to amino acid sequencing. An amino acid sequence motif for the phosphorylation site of the kinase within a cyclic peptide is determined based upon the relative abundance of amino acid residues at each degenerate position of the peptide library.

The method of the invention can be used to determine an amino acid sequence motif for the phosphorylation site of any kinase. For example, the kinase can be a protein-serine/threonine specific kinase (in which case a library with a fixed non-degenerate serine or threonine is used), a protein-tyrosine specific kinase (in which case a library with a fixed non-degenerate tyrosine is used) or a dual-specificity kinase (in which case a library with either a fixed non-degenerate serine, threonine or tyrosine can be used). Nonlimiting examples of protein kinases which are encompassed by the invention can be found in Hanks et al. (1988) Science 241:42-52.

Protein-serine/threonine specific kinases encompassed by the invention include: 1) cyclic nucleotide-dependent kinases, such as cyclic-AMP-dependent protein kinases (e.g., protein kinase A) and cyclic-GMP-dependent protein kinases; 2) calcium-phospholipid-dependent kinases, such as protein kinase C; 3) calcium-calmodulin-dependent kinases, including CaMII, phosphorylase kinase (PhK), myosin light chain kinases (e.g., MLCK-K, MLCK-M), PSK-H1 and PSK-C3; 4) the SNF1 family of protein kinases (e.g., SNF 1, nim1, KIN1 and KIN2);5) casein kinases (e.g., CKII);6) the Raf-Mos proto-oncogene family of kinases, including Raf, A-Raf, PKS and Mos; and 7) the STE7 family of kinases (e.g. STE7 and PBS2). Additionally, the protein-serine/threonine specific kinase can be a kinase involved in cell cycle control. Many kinases involved in cell cycle control have been identified. Cell cycle control kinases include the cyclin dependent kinases, which are heterodimers of a cyclin and kinase (such as cyclin B/p33^{cdc2}, cyclin A/p33^{CDK2}, cyclin E/p33^{CDK2} and cyclin D1/p33^{CDK4}). Other cell cycle control kinases include Wee1 kinase, Nim1/Cdr1 kinase and Wis1 kinase.

Protein-tyrosine specific kinases encompassed by the invention include: 1) members of the src family of kinases, including pp60^{c-src}, pp60^{v-src}, Yes, Fgr, FYN, LYN, LCK, HCK, Dsrc64 and Dsrc28; 2) members of the Ab1 family of kinases, including Ab1, ARG, Dash, Nab1 and Fes/Fps; 3) members of the epidermal growth factor receptor (EGFR) family of kinases, including EGFR, v-Erb-B, NEU and DER; 4) members of the insulin receptor (INS.R) family of growth factors, including INS.R, IGF1R, DILR, Ros, 7less, TRK and MET; 5) members of the platelet-derived growth factor receptor (PDGFR) family of kinases, including PDGFR, CSF1R, Kit and RET.

Other protein kinases which can be used in the method of the invention include syk, ZAP70, Focal Adhesion Kinase, erk1, erk2, erk3, MEK, CSK, BTK, ITK, TEC, TEC-2, JAK-1, JAK-2, LET23, c-fms, S6 kinases (including p70^{S6} and RSKs), TGF-β/activin receptor family kinases and Clk.

The amino acid sequence motifs determined by the method of the invention are useful for predicting whether a protein is a substrate for a particular protein kinase. The primary amino acid sequence of a known protein can be examined for the presence of the determined amino acid sequence motif. If the same or a very similar motif is present in the protein, it can be predicted that the protein could be a substrate for that protein kinase.

B. Phosphatases as Binding Compounds

In another embodiment, a binding compound used in the method is a phosphatase. When the binding compound is a phosphatase, the ODCPL is constructed such that it contains a phosphorylated amino acid residue at a fixed, non-degenerate positions. This phosphorylated amino acid residue then becomes dephosphorylated by the phosphatase. In a preferred embodiment, an ODCPL for use with serine, threonine or tyrosine phosphatases comprises cyclic peptides comprising a formula:

[chemical structure]

wherein Zₐₐ is a non-degenerate phosphorylated amino acid selected from the group consisting of phosphoserine, phosphothreonine and phosphotyrosine, Xₐₐ is any natural or unnatural αamino acid, R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive. Preferably, Zaa is the only phosphorylated amino acid within the cyclic peptides.

For phosphatases, the incubation, separation and linearization steps are performed in as similar manner as described above for kinases except that: the phosphatase and the ODCPL are incubated under conditions that allow for dephosphorylation of library members by the phosphatase and in the separation step, nonphosphorylated peptides are selected (rather than phosphorylated peptides). Sequencing and determination of the amino acid motif for the dephosphorylation site are performed as described above for kinases.

C. SH2 and SH3 Domains as Binding Compounds

In yet another embodiment, a binding compound used in the method comprises a Src Homology 2 (SH2) domain or a Src Homology 3 (SH3) domain. It is known that SH2 domains have specificity for phosphotyrosine-containing targets. Accordingly, when a binding compound comprises an SH2 domain, the ODCPL is designed to contain a phosphotyrosine residue at a fixed, nondegenerate position. In a preferred embodiment, an ODCPL for use with SH2 domains comprises cyclic peptides comprising a formula:

[chemical structure]

wherein Zₐₐ is a non-degenerate phosphotyrosine residue, Xₐₐ is any natural or unnatural αamino acid, R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive.

In a preferred embodiment, the binding compound comprising an SH2 domain is immobilized on a solid support, for example by expressing the SH2 domain as a GST fusion protein and immobilizing the fusion protein on glutathione-agarose. To select for library members that interact with the SH2 domain, the ODCPL is passed over the solid support to allow the binding compound to interact with a subpopulation of library members capable of interacting with the SH2 domain, and library members incapable of interacting with the SH2 domain are washed away to thereby separate the subpopulation of library members capable of interacting with the SH2 domain from library members incapable of interacting with the SH2 domain. Methods for preparing SH2-GST fusion proteins and for screening linear peptide libraries against these fusion proteins are described further in Songyang et al. (1993) Cell 72:767-778. These methods can be adapted for use in the cyclic peptide screening methods of the invention.

It is known that SH3 domains preferentially bind to proline-containing targets. Accordingly, when the binding compound comprises an SH3 domain, the ODCPL preferably contains at least one proline residue at a fixed, nondegenerate position. In a preferred embodiment, an ODCPL for use with SH3 domains comprises cyclic peptides comprising a formula:

[chemical structure]

wherein Zₐₐ is a non-degenerate proline, Xₐₐ is any natural or unnatural α-amino acid, R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive.

Preferably, the binding compound comprising an SH3 domain is immobilized on a solid support, as described above for SH2 domains. The ODCPL then is passed over the solid support to allow the binding compound to interact with a subpopulation of library members capable of interacting with the SH3 domain, and library members incapable of interacting with the SH3 domain are washed away to thereby separate the subpopulation of library members capable of interacting with the SH3 domain from library members incapable of interacting with the SH3 domain.

Linearization of the selected cyclic peptides, sequencing and determination of the amino acid sequence motif are performed as described above for kinases.

D. Proteases as Binding Compounds

In yet another embodiment, the binding compound is a protease. If the particular protease is known to preferentially cleave at a certain amino acid residue(s), the ODCPL can be designed to incorporate the preferred amino acid residue(s) at a fixed, non-degenerate position. For example, members of the caspase family of cysteine proteases are known to preferentially cleave after an aspartic acid residue (although the substrate specificity of particular caspases is influenced by amino acid residues surrounding the aspartic acid residue). Accordingly, when the binding compound is a caspase, the ODCPL is designed to have an aspartic acid residue at a fixed, non-degenerate position. Alternatively, proteases which cleave peptide bonds include TNF-alpha (Tumor Necrosis Factor) converting enzymes which cleave Ala-Val bonds and serine proteases, such as chymotrypsin which preferentially cleaves the bond after an aromatic residue and trypsin which preferentially cleaves after arginine and lysine residues.

Proteases which cleave specific peptide bonds include aspartyl proteases such as pepsin and cathepsin D. Proteases which belong to the metalloproteinase family include collagenase, stromeylsin, angiotensin-converting enzyme, fertilin-α and β, and the MMP (matrix-metalloproteinase) class. Suitable cysteine proteases include papain, cathepsin B 1, B2, H, L and F. The aminopeptidase family includes aminopeptidases A, N, GP160 and leucine aminopeptidase. Additionally, the carboxypeptidase family includes carboxypeptidases H, M and E.

When the binding compound is a protease, the subpopulation of library members that is capable of interacting with the protease is linearized by allowing the protease to cleave these library members (e.g., the linearization step is achieved using the protease itself). Thus, within the library, those library members that become linearized represent library members that are capable of interacting with the protease.

Example 3 further describes application of the method of the invention to proteases.

E. Antibodies as Binding Compounds

In yet another embodiment, the binding compound is an antibody, or antigen-binding fragment thereof. Examples of antigen-binding fragments include Fab, Fd, Fv, F(ab′)₂ and scFv fragments. The screening method of the invention can be used to map the epitope(s) of a monoclonal antibody, described further in Example 6. Moreover, the invention provides methods for generating monoclonal antibodies (e.g., for diagnostic or therapeutic use) using cyclic peptide libraries as immunogens, described further in Example 7.

F. Other Binding Compounds

The screening methods of the invention are applicable to a wide variety of additional binding compounds. Non-limiting examples include WW domains, PTB domains, PDZ domains, LIM domains, pleckstrin homology domains, zinc finger domains, extracellular growth factors, growth factor receptors, adhesion molecules, intercellular signaling molecules, lipid phosphatases, 7-transmembrane receptor proteins, proteases, ion channels, methyltransferases, ubiquitinating enzymes and peptidyl-transferases.

When information is already known about the binding preferences of a particular binding compound, this information can be utilized in the design of the ODCPL. For example, for WW domains, which are known to preferentially bind proline-rich regions, the ODCPL can be designed to incorporate at least one proline residue at a fixed, non-degenerate positions. For PTB domains, which have been reported to preferentially bind an NPXpY motif (wherein, N is asparagine, P is proline, X is any amino acid and pY is phosphotyrosine), this motif can be incorporated into the ODCPL. For PDX domains, which have been reported to preferentially bind an (S/T)XV motif (wherein S is serine, T is threonine, X is any amino acid and V is valine), this motif can be incorporated into the ODCPL.

G. Other Embodiments

Another embodiments of the screening method of the invention include use of the cyclic peptide libraries in combination with linear peptide libraries. That is, the same binding compound can be screened against both a cyclic peptide library and a linear peptide library. Use of cyclic peptide libraries in combination with linear peptide libraries for epitope mapping of monoclonal antibodies is described further in Example 6.

In yet another embodiment of the screening methods of the invention, the binding compound is encapsulated in a liposome prior to contacting the binding compound with the ODCPL (described further in Example 5). By encapsulating the binding compound within a liposome prior to peptide library screening, only those cyclic peptides which are permeable to lipid bilayers (and therefore good drug candidates and candidate lead compounds) can enter the liposome and interact with the binding compound. Thus, by combining the cyclic library screening with the lipid encapsulation of the binding compound, the search for cyclic-peptide-based drug candidates is converted into a one-step screen that incorporates both optimal substrate selection and membrane permeability.

Still another embodiment of the screening method provides for duplex screening of both linear and cyclic libraries to identify optimal chain length or ring size for a particular binding compound. This embodiment utilizes the “tag termination technique” (TTT), which is described in further detail in Example 8.

II. Oriented Degenerate Cyclic Peptide Libraries

Another aspect of the invention pertains to oriented degenerate cyclic peptide libraries. In a preferred embodiment, the invention provides and ODCPL which comprises cyclic peptides comprising a formula:

[chemical structure]

wherein Zₐₐ is a non-degenerate natural or unnatural α-amino acid, Xₐₐ is any natural or unnatural α-amino acid, R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive. In other embodiment, m and n, independently, are selected from 1-7 inclusive. In yet another embodiment, m and n, independently, are selected from 1-5 inclusive. In a preferred embodiment, R—R′ is methionine-alanine. In various embodiments, Zₐₐ can be, for example, a non-degenerate phosphorylatable amino acid (such as serine, threonine or tyrosine), non-degenerate phosphorylated amino acid (such as phosphoserine, phosphothreonine or phosphotyrosine), a proline, a nonnatural α-amino acid, a hydrophobic natural or nonnatural α-amino acid or a hydrophilic natural or nonnatural α-amino acid.

Preferably the cyclic peptides of the ODCPL are cyclized in a “head-to-tail” manner by formation of a peptide bond between the amino and carboxy terminii. Methods for solid-phase synthesis of “head-to-tail” cyclic peptides have been described (see e.g., McMurray, J. S. (1991) Tetrahedron Letter 32:7679-7682; Trzeciak, A. and Bannwarth, W. (1992) Tetrahedron Letters 33:4557-4560; Tromelin, A. et al. (1992) Tetrahedron Letters 33:5197-5200; PCT Publication WO 95/34577). Methods for synthesizing cyclic peptides are described further in Example 1.

The cyclic peptides libraries of the invention contain at least one fixed, nondegenerate amino acid positions and several degenerate amino acid positions. In a preferred embodiment, the amino acid residues on either side of the fixed non-degenerate amino acid residue are degenerate (e.g., immediately N-terminal and C-terminal to the non-degenerate residue), thus enabling one to determine an interaction site motif for the region surrounding the fixed amino acid residue. For example, four amino acid residues located on each side of the non-degenerate amino acid residue can be degenerate (e.g., positions −4, −3, −2, −1, +1, +2, +3 and +4, relative to the non-degenerate amino acid residue at position 0, can be degenerate). The degenerate positions in the peptides of an oriented degenerate cyclic peptide library can be created such that any one of the twenty natural amino acids, as well as unnatural α-amino acids, can occupy those positions. However, in order to reduce “background” events (e.g., enzymatic events at a residue other than a fixed residue), it is preferred that the degenerate positions not contain amino acid residues that can be acted upon by the particular binding compound being examined. Thus, for example, when the binding compound is a protein-serine/threonine kinases and the fixed residue is a serine or threonine, it is preferred that the degenerate positions not contain serine or threonine. Likewise, for a protein-tyrosine specific kinase, where the fixed residue is a tyrosine, it is preferred that the degenerate positions not contain tyrosine.

However, if desired, an oriented degenerate cyclic peptide library can contain residues in addition to the fixed residue that can be acted upon by the binding compound, since it is possible to estimate the degree of background which will occur when using such a library and take this background into consideration when evaluating the results of the library screening. For example, consider the problem of including Tyr at the degenerate positions of a library having 8 degenerate positions that is to be used with a protein-Tyr kinase. The kinase may phosphorylate the tyrosine residues at the degenerate positions as well as the Tyr at the fixed position. The theoretical fraction of peptides with Tyr at one of the degenerate sites in addition to the fixed site (assuming a degeneracy of 20 at each residue) is 8×(1/20)=0.4. Thus, about 40% of the phosphopeptides purified are likely to be phosphorylated at the wrong residue. It is possible to get a good estimate of the extent of this problem since those peptides that are phosphorylated at sites other than the fixed residue will have detectable non-phosphorylated Tyr at the sequencing cycle corresponding to the fixed residue when the sequencing is performed on the mixture. Although these phosphopeptides will cause a background problem when the data are plotted, the problem is less serious than initially implicated from the 40% contamination assumed. For example, consider the case in which the protein-tyrosine kinase being evaluated phosphorylates the sequence Glu-Xxx-Tyr and the fixed Tyr is at position 7 in the peptides. Those peptides with Glu at position 5 will be preferentially phosphorylated. However, peptides with Glu at position 3 and Tyr at position 5 will also be phosphorylated and appear in the mixture. Similarly, those with Glu at 4 and Tyr at 6, Glu at 6 and Tyr at 8, Glu at 8 and Tyr at 10 and Glu at 9 and Tyr at 11 will also be selected. Each of these subfamilies of peptides is far less abundant than the group with Tyr fixed at position 7 (in theory, 1/20th). Thus, the expected result is that Glu will be very abundant at cycle 5 but will also be somewhat elevated (1/20th as much as in cycle 5) at cycles 3, 4, 6, 8, and 9. In general this background is unlikely to be a problem. The importance of re-evaluating protein kinases with phosphorylatable residues at the degenerate positions is that Ser, Thr or Tyr residues upstream or downstream of the phosphorylated residue are already known to be important for some protein kinases.

Additionally, certain amino acid residues may be omitted from a degenerate position in an oriented degenerate cyclic peptide library for practical reasons. For example, tryptophan and cysteine residues may be omitted from the degenerate positions because there can be problems with detecting these residues during amino acid sequencing and cysteine residues may cause peptide dimer formation. However, a peptide library containing Trp and Cys at a degenerate position can be used in the method of the invention. For example, a “second generation” library can be made based upon the amino acid sequence motif determined from initially screening a protein kinase with a peptide library which does not contain Trp or Cys at degenerate positions. At each degenerate position of this library, the peptides would have either the preferred amino acid residue determined from the initial screening or Cys or Trp. Thus, in this library, rather than being 1 of 20 possible natural residues at the degenerate position, Cys and Trp would be 1 of 3 possible residues at the degenerate position. Therefore, Trp and Cys will have a much stronger signal during amino acid sequencing of the peptides, thereby allowing for detection of these residues and for evaluation of their influence on the amino acid sequence motif of the phosphorylation site.

A preferred type of oriented degenerate cyclic peptide library for use in the method of the invention is a soluble synthetic peptide library. The term “soluble synthetic peptide library” is intended to mean a population of peptides which are constructed by in vitro chemical synthesis, for example using an automated peptide synthesizer, and which are not connected to a solid support such as a bead or a cell. For general descriptions of the construction of soluble synthetic peptide libraries see for example Houghten, R. A., et al., (1991) Nature 354:84-86 and Houghten, R. A., et al., (1992) BioTechniques 13:412-421. Standard techniques for in vitro chemical synthesis of peptides are known in the art. For example, peptides can be synthesized by (benzotriazolyloxy)tris(dimethylamino)-phosphonium hexafluorophosophate (BOP)/1-hydroxybenzotriazole coupling protocols. Automated peptide synthesizers are commercially available (e.g., Milligen/Biosearch 9600). To create degenerate positions within peptides of a soluble synthetic peptide library, two approaches can be used. A preferred approach is to divide the resin upon which the peptides are synthesized into equivalent portions and then couple each aliquot to a different amino acid residue to create a degenerate position. After this coupling, the resin aliquots are recombined and the procedure is repeated for each degenerate position. This approach results in approximately equivalent representation of each different amino acid residue at the degenerate position. Alternatively, a mixture of different amino acid residues can be added to a coupling step to create a degenerate position. However, different amino acid residues have different coupling efficiencies and therefore if equal amounts of each amino acid are used, each amino acid residue may not be equivalently represented at the degenerate position. The different coupling efficiencies of different amino acids can be compensated for by using a “weighted” mixture of amino acids at a coupling step, wherein amino acids with lower coupling efficiencies are present in greater abundance than amino acids with higher coupling efficiencies.

An alternative type of oriented degenerate cyclic peptide library which can be used in the method of the invention is a solid-support bound peptide library. The term “solid-support bound peptide library” is intended to mean a population of peptides which are connected to a solid support such as a bead or plastic pin. For general descriptions of the construction of solid-support bound peptide libraries see for example Geysen, H. M., et al. (1986) Mol. Immunol. 23:709-715; Lam, K. S., et al. (1991) Nature 354:82-84; and Pinilla, C., et al. (1992) BioTechniques 13:901-905. For this type of library, the peptides are synthesized attached to the solid support, such as a bead, and degenerate positions are created by splitting the population of beads, coupling different amino acids to different subpopulations and recombining the beads. The final product is a population of beads each carrying many copies of a single unique peptide. Thus, this approach has been termed “one bead/one peptide”.

A soluble synthetic peptide library is strongly preferred for use in the method of the invention because the bulk population of isolated peptides can be sequenced simultaneously, thus directly providing information on the relative abundance of different amino acid residues at each degenerate position within the population of peptides. In contrast, if a solid-support bound library is used, each isolate gives only the single amino acid sequence of that isolated peptide and therefore many isolated peptides must be individually sequenced before one can arrive at a consensus motif. Even then, it is not possible to predict the order of affinities of the various peptides isolated unless they are individually synthesized and compared in binding experiments. One cannot be certain that one has not missed the best possible motif because not enough isolated peptides were sequenced. In addition, while the soluble synthetic oriented degenerate cyclic peptide library provides predictions about substitutions that would severely reduce the affinity of the peptide, no such information can be obtained from a solid-support bound library.

The diversity of the peptide library (e.g., the number of different peptides contained within the library) is a function of the number of degenerate residues: the greater the number of degenerate residues the greater the diversity. For example, a library in which only 2 positions are degenerate and any of the twenty natural amino acid can be at these degenerate positions would represent 400 unique peptides (20²) whereas a library in which 8 positions are degenerate and any amino acid can be at these positions would represent approximately 2.5×10¹⁰ unique peptides (20⁸). An oriented degenerate peptide library can be prepared in which the degenerate residues encompass the region likely to interact with the binding compound. The number of amino acids residues that influence the substrate specificity of a binding compound will differ for different binding compounds. Thus, it is expected that oriented degenerate cyclic peptide libraries which will be useful in the method of the invention may have as few as one degenerate amino acid residue to as many as ten degenerate amino acid residues on either side of the fixed residue. Alternative to having an equal number of degenerate residues on either side of the fixed residue, one can use a library which has unequal numbers of degenerate residues on either side of the fixed residue (e.g., 2 on one side and 4 on the other, etc.). The optimal length (and corresponding optimal ring size) for an ODCPL for a particular binding compound can be determined using methods described further in Example 8.

III. Methods for Purifying Cyclic Peptides

Yet another aspect of the invention pertains to methods for purifying cyclic peptides from linear peptides. The method of the invention involves:

providing a mixture of cyclic peptides and linear peptides;

contacting the mixture with a blocking agent that reacts with the free amino termini of the linear peptides to form amino-protected linear peptides;

contacting the mixture with a binding agent that is capable of interacting with the amino-protected linear peptides but incapable of interacting with the cyclic peptides; and

separating the amino-protected linear peptides from the cyclic peptides to thereby purify the cyclic peptides.

In a preferred embodiment, the blocking agent is a biotin, which biotinylates the free amino-termini of the linear peptides, and the binding agent is a biotin-binding agent, such as avidin or streptavidin. For example, biotinylated linear peptides can be separated from cyclic peptides by passing the mixture over an avidin column. Methods for purifying cyclic peptides using biotin-avidin affinity chromatography are described further in Example 2. In an alternative embodiment, the binding agent is an antibody that is specific for the blocking agent and the cyclic peptides are purified using antibody affinity chromatography.

This invention is further illustrated by the following examples which should not be construed as limiting. The contents of all references and published patents and patent applications cited throughout the application are hereby incorporated by reference.

### EXAMPLE 1

On Resin Head-to-Tail Cyclization of Peptides using Asp α-Allyl Esters

It has been shown that allyl based protecting groups can be removed under mild conditions by Pd(0) catalyzed allyl transfer (H. Kunz & A. Waldmann (1983) Angew. Chem. Int. Ed. Eng., 23:71; H. Kunz & C. Unverzagt (1983) Angew. Chem. Int. Ed. Eng., 23:436; P. D. Jeffrey & S. W. McCombie (1982) J. Org Chem., 47:587; B. M. Trost (1980) Acc. Chem. Res., 13:385). Following resin coupling of the carboxylic acid side chain of the FMOC α-allyl ester of Asp, the degenerate peptide library containing an orienting residue (Y of FIG. 1 as shown) is synthesized using standard FMOC-amino acid chemistry. Following synthesis, the FMOC protecting group on the Met residue is removed with piperidine, the allyl ester removed by Barany's method and head-to-tail cyclization achieved by activation of the free resin-bound carboxyl of Asp by the coupling reagent HBTU. Un-cyclized peptides are blocked by biotinylation, followed by final side chain deprotection and resin cleavage.

More specifically, the carboxylic acid side chain of Fmoc alpha-allyl esters of Asp is first coupled to the resin (NovaSyn TGR resin, 0.25 mmol) and the degenerate peptide library is then synthesized according to the standard Fastmoc cycles with HBTU (2-(1H-Benzotriazole-1-y1)-1,1,3,3-tetramethyluronium hexafluorophosphate) coupling protocols on a Peptide Biosynthesizer (Minipore Model). At the degenerate positions, 18 different Nα-FMOC-blocked amino acids (excluding Trp, Cys) are added simultaneously in four-fold molar excess of the coupling resin. Prior to cyclization, the Fmoc group on Met is removed and the allyl ester subsequently deprotected by Barany's method (F. Albericio et al. in “Peptides 1992”, C. H. Schnelder & A. N. Eberle (eds), ESCOM, Leiden, 1993, pp191; S. A. Kates et al. in “Peptides; Chemistry, Structure & Biology”, R. S. Hodges & J. A. Smith (eds), ESCOM, Leiden, 1994, pp 113).

As illustrated in FIG. 1, cyclization occurs upon activation of the free resin-bound carboxy group of Asp by the coupling reagent, HBTU, over 5 hours. Cyclization is through the reaction of the methionines' free α-amino group with the aspartic acids' carboxyl ends. The uncyclized peptides are then blocked by biotin under standard reaction conditions. The resin bound cyclic library mixture is then biotinylated in the following manner: biotin (0.8 mmol) was dissolved in a 1:1 mixture of DMSO containing 0.8 mmol each of N-methylmorpholine and 1-benzotriazolyloxy-tris-dimethylamio-phosphonium hexafluorophosphate/0.65M diisopropylethylamine containing 0.8 mmol N-hydroxybenzotriazole and allowed to react with the resin-bound cyclized library overnight at room temperature. Final deprotection of the peptides and their release from the resin by TFA gave 33 mg of the crude peptides as a white solid. The TFA cleavage conditions were: 2 ml out of mixture of 10 ml TFA, 0.4 ml water, 0.5 ml thioanisol, 0.75 g phenol and 0.25 ml ethanol diphiol, room temperature for 3 hours. The MALDI-TOF-MS (Matrix-Assisted-Laser-Desorption-Ionization Time-of-Flight Mass Spectrometry Mass Spectrum) (on Perseptive Biosystem Voyager Linear System) showed the expected major broad peak (@ MW 1778) and three other minor species (@ MW 3623, 6503, 7352 respectively).

### EXAMPLE 2

Purification of the Cyclic Peptide Library by Avidin-Biotin Affinity Chromatography

After the on-resin intra-molecular cyclization reaction, the uncyclized peptides with free amino terminals were derivatised with biotin (described in Example 1). Purification of the cyclic peptide library, illustrated in FIG. 2A, was performed on immobilized avidin following established procedures. (Quesnel, A. (1995) Analytical Biochemistry 231:182-187). The unwanted biotinylated linear peptides were retained in the avidin column. Briefly, the crude mixture was dissolved in phosphate buffer (PBS, 10 mM phosphate, 150 mM NaCl, 5 mM KCl, pH 7.2) and applied to a column of immobilized avidin (SIGMA A-9207; insolubilized on cross-linked 6% beaded agarose; 10 ml suspension with 3.6 mg avidin per ml of packed gel; packed in 10×1 cm column;) with a flow rate of 0.6 ml/min. The column was washed with 10-15 bed volumes of phosphate buffer at 4° C. The eluents were collected and dried and the peptide mixture desalted on a Sephadex G-25 (1.5×85 cm) column using water as eluent. Peptide elution was detected by monitoring absorbance @ 280 nm. 1 ml fractions were collected and those containing peptide were pooled and lyophilized to yield ca. 7.0 mg of white solid.

The initial products of the on-resin cyclization approach described in FIG. 1 were characterized by matrix-assisted laser-desorption ionization time-of-flight mass spectroscopy (MALDI-TOF-MS) using a Perspective Biosystem Voyager Linear System instrument. This revealed the expected broad peak at Mw. 1778, and three other minor species at Mws. 3623, 6503, and 7352 (FIG. 2B). These latter peaks are not cyclic, and result from dimerization and oligomerization reactions between individual resin-bound chains during the cyclization process. Following cyclization, the unwanted linear peptides and oligomers were removed by derivitization of the library mixture with biotin, followed by chromatography on an immobilized avidin column. The unwanted biotinylated peptides were retained, while the desired cyclic library product was eluted with phosphate-buffered saline. The eluents were subsequently desalted on a Sephadex G-25 column using water as the eluent. Mass spectroscopy (MALDI-TOF-MS) showed a single peak of Mw at 1715 (FIG. 2C), with successful removal of the three contaminants of higher weight by this technique.

TABLE I
Recovery of each amino acid (pmoles) in cyclic peptide library following
CNBr cleavage and amino acid analysis

| | A | R | N | D | C | E | Q | G | H | I |
|---|---|---|---|---|---|---|---|---|---|---|
| Cycle | ALA | ARG | ASN | ASP | CYS | GLU | GLN | GLY | HIS | ILE |
| 1 | 2205.9 | 36.5 | 0 | 2822.4 | 0 | 106.7 | 11.9 | 35.3 | 71.4 | 14.9 |
| 2 | 560.2 | 271.4 | 30.7 | 50 | 0 | 186.5 | 38.9 | 233.6 | 18.4 | 53.5 |
| 3 | 362.6 | 330.8 | 37.4 | 50 | 0 | 215.2 | 58.2 | 278.2 | 22.7 | 71.9 |
| 4 | 224.6 | 203.1 | 51.2 | 50 | 0 | 146.3 | 54.2 | 183.3 | 18.6 | 44.2 |
| 5 | 173.2 | 179 | 64.6 | 54 | 0 | 142.1 | 41.2 | 171.5 | 20.5 | 46.6 |
| 6 | 146.1 | 110.7 | 55 | 45.7 | 0 | 60.2 | 17.6 | 85.1 | 11.9 | 22.5 |
| 7 | 165.7 | 134.5 | 55 | 83.6 | 0 | 86.1 | 28.2 | 119.9 | 14 | 30.7 |
| 8 | 178.7 | 139.8 | 41.2 | 73.3 | 0 | 78.8 | 25.2 | 128.8 | 19.8 | 35.3 |
| 9 | 184.6 | 175.3 | 57.9 | 73.1 | 0 | 77.9 | 27.2 | 110.6 | 21.7 | 38.5 |
| 10 | 121.8 | 134.2 | 47.3 | 57 | 0 | 49 | 18 | 75.6 | 13.8 | 30.7 |
| 11 | 456.2 | 93.4 | 38.3 | 32.7 | 0 | 24 | 8.2 | 39.3 | 6.3 | 12.1 |
| 12 | 233.6 | 371.8 | 40.6 | 16.2 | 0 | 15 | 5.6 | 26.5 | 4.8 | 7.5 |
| 13 | 76.1 | 336.8 | 30.8 | 10.1 | 0 | 8.4 | 3.2 | 17.9 | 3.3 | 3.5 |
| 14 | 63 | 173.2 | 202.2 | 27.6 | 0 | 8.4 | 3.3 | 16.5 | 2.4 | 4.3 |
| 15 | 42 | 77.1 | 99.6 | 15.9 | 0 | 7.3 | 2.9 | 16.1 | 2.1 | 4.2 |

| | L | K | M | F | P | S | T | W | Y | V |
|---|---|---|---|---|---|---|---|---|---|---|
| Cycle | LEU | LYS | MET | PHE | PRO | SER | THR | TRP | TYR | VAL |
| 1 | 28.4 | 18.8 | 89.4 | 34.4 | 39.7 | 37.6 | 3678.6 | 2.5 | 125.3 | 36.3 |
| 2 | 86.6 | 49.9 | 29.7 | 113.8 | 110.1 | 91 | 75 | 1.6 | 201.8 | 113.3 |
| 3 | 109.2 | 65.6 | 30 | 139.2 | 111.5 | 117.9 | 103.6 | 1.1 | 222.7 | 138.5 |
| 4 | 73.3 | 58.1 | 18.8 | 98.1 | 71.3 | 81.8 | 59.8 | 0.4 | 170.1 | 90.6 |
| 5 | 66.5 | 57.3 | 27.9 | 94.3 | 69.6 | 80.4 | 53.6 | 0.2 | 148.3 | 90.5 |
| 6 | 31.9 | 27.5 | 23.1 | 50.8 | 31.4 | 31.4 | 29.2 | 0.6 | 1050.9 | 45.5 |
| 7 | 43.8 | 44 | 27.2 | 86.4 | 38.4 | 53.4 | 39.1 | 0.1 | 287.1 | 63.5 |
| 8 | 60.4 | 41 | 29.9 | 71.9 | 40 | 53.4 | 38.1 | 0.2 | 123.5 | 73.7 |
| 9 | 63.2 | 40.5 | 26.8 | 74.5 | 46.4 | 55.6 | 41.9 | 0.3 | 92.6 | 73.5 |
| 10 | 34.1 | 30.6 | 21.1 | 48.6 | 35.4 | 36.7 | 26.4 | 0.3 | 56.3 | 55.5 |
| 11 | 14.1 | 13.2 | 15 | 19.5 | 16.9 | 16.2 | 12.6 | 0.1 | 33 | 22.8 |
| 12 | 9 | 8.5 | 15.1 | 12 | 11.4 | 10.4 | 9 | 0.1 | 29.4 | 13.7 |
| 13 | 5.7 | 4.8 | 11 | 7.1 | 6.3 | 6.1 | 5.7 | 0.1 | 19.2 | 8 |
| 14 | 6.4 | 4.9 | 11.2 | 7.2 | 6 | 6.1 | 5.7 | 0.1 | 21.5 | 7.7 |
| 15 | 5.4 | 4.4 | 61.1 | 6.8 | 6 | 5.6 | 16.4 | 0.1 | 17.4 | 7.2 |

TABLE II
Mole percentages of each amino acid per cycle following
CNBr cleavage and sequencing of the cyclic peptide library

| | A | R | N | D | C | E | Q | G | H | I |
|---|---|---|---|---|---|---|---|---|---|---|
| Cycle | ALA | ARG | ASN | ASP | CYS | GLU | GLN | GLY | HIS | ILE |
| 1 | 0.736 | 0.012 | 0 | 0.016 | 0 | 0.035 | 0.003 | 0.011 | 0.023 | 0.049 |
| 2 | 0.244 | 0.118 | 0.013 | 0.021 | 0 | 0.081 | 0.016 | 0.102 | 0.008 | 0.023 |
| 3 | 0.150 | 0.137 | 0.015 | 0.020 | 0 | 0.089 | 0.024 | 0.114 | 0.009 | 0.029 |
| 4 | 0.132 | 0.119 | 0.030 | 0.029 | 0 | 0.861 | 0.031 | 0.107 | 0.010 | 0.026 |
| 5 | 0.109 | 0.113 | 0.040 | 0.034 | 0 | 0.069 | 0.026 | 0.108 | 0.012 | 0.029 |
| 6 | 0.077 | 0.058 | 0.029 | 0.024 | 0 | 0.032 | 0.009 | 0.045 | 0.006 | 0.011 |
| 7 | 0.120 | 0.097 | 0.039 | 0.060 | 0 | 0.062 | 0.020 | 0.086 | 0.010 | 0.022 |
| 8 | 0.142 | 0.111 | 0.032 | 0.058 | 0 | 0.062 | 0.020 | 0.102 | 0.015 | 0.026 |
| 9 | 0.144 | 0.136 | 0.045 | 0.057 | 0 | 0.060 | 0.021 | 0.066 | 0.016 | 0.030 |
| 10 | 0.136 | 0.150 | 0.053 | 0.063 | 0 | 0.054 | 0.020 | 0.084 | 0.015 | 0.034 |
| 11 | 0.521 | 0.107 | 0.043 | 0.037 | 0 | 0.027 | 0.009 | 0.045 | 0.007 | 0.013 |
| 12 | 0.277 | 0.442 | 0.048 | 0.019 | 0 | 0.017 | 0.006 | 0.031 | 0.005 | 0.008 |
| 13 | 0.134 | 0.597 | 0.054 | 0.017 | 0 | 0.014 | 0.005 | 0.031 | 0.005 | 0.006 |
| 14 | 0.093 | 0.304 | 0.354 | 0.048 | 0 | 0.014 | 0.005 | 0.032 | 0.004 | 0.007 |
| 15 | 0.105 | 0.193 | 0.250 | 0.039 | 0 | 0.018 | 0.007 | 0.040 | 0.005 | 0.010 |

| | L | K | M | F | P | S | T | W | Y | V |
|---|---|---|---|---|---|---|---|---|---|---|
| Cycle | LEU | LYS | MET | PHE | PRO | SER | THR | TRP | TYR | VAL |
| 1 | 0.009 | 0.006 | 0.029 | 0.011 | 0.013 | 0.012 | 0.016 | 0 | 0.0421 | 0.012 |
| 2 | 0.037 | 0.021 | 0.012 | 0.049 | 0.048 | 0.039 | 0.021 | 0 | 0.0881 | 0.049 |
| 3 | 0.045 | 0.027 | 0.012 | 0.057 | 0.046 | 0.048 | 0.020 | 0 | 0.0924 | 0.057 |
| 4 | 0.043 | 0.342 | 0.011 | 0.057 | 0.042 | 0.046 | 0.035 | 0 | 0.1002 | 0.053 |
| 5 | 0.042 | 0.038 | 0.017 | 0.059 | 0.044 | 0.050 | 0.033 | 0 | 0.0937 | 0.057 |
| 6 | 0.017 | 0.014 | 0.012 | 0.027 | 0.016 | 0.016 | 0.015 | 0 | 0.5600 | 0.024 |
| 7 | 0.031 | 0.031 | 0.019 | 0.048 | 0.027 | 0.038 | 0.028 | 0 | 0.2079 | 0.045 |
| 8 | 0.048 | 0.032 | 0.023 | 0.057 | 0.031 | 0.042 | 0.030 | 0 | 0.0985 | 0.058 |
| 9 | 0.049 | 0.031 | 0.020 | 0.058 | 0.036 | 0.043 | 0.032 | 0 | 0.0722 | 0.057 |
| 10 | 0.038 | 0.034 | 0.023 | 0.054 | 0.039 | 0.041 | 0.029 | 0 | 0.0631 | 0.062 |
| 11 | 0.016 | 0.015 | 0.017 | 0.022 | 0.019 | 0.018 | 0.014 | 0 | 0.0378 | 0.026 |
| 12 | 0.010 | 0.010 | 0.017 | 0.014 | 0.013 | 0.012 | 0.010 | 0 | 0.035 | 0.016 |
| 13 | 0.010 | 0.008 | 0.019 | 0.012 | 0.011 | 0.010 | 0.010 | 0 | 0.0340 | 0.014 |
| 14 | 0.011 | 0.008 | 0.019 | 0.012 | 0.010 | 0.010 | 0.010 | 0 | 0.0377 | 0.013 |
| 15 | 0.013 | 0.011 | 0.153 | 0.017 | 0.015 | 0.014 | 0.041 | 0 | 0.0437 | 0.018 |

The purified cyclic library product was then subjected to cleavage using CNBr and the resulting mixture of linear peptides was subjected to automated peptide sequencing (Edman degradation followed by standard 2-buffer column chromatography) using an Applied Biosystems Model 477A peptide sequencer. Tables I and II show the recovery of each amino acid during 15 cycles of sequencing, and the mole-percentages of each amino acid at each cycle. The large values of Asp and Thr in the first few cycles are artifactual, resulting from sample and buffer impurities. As expected from the CNBr cleavage following methionine, the dominant amino acid in the first cycle is alanine, the predominant amino acid cycle in 6 is the orienting residue Tyr, and the predominant amino acids at cycles 11-14 correspond to the sequence Ala-Arg-Arg-Asn (see FIG. 1).

### EXAMPLE 3

Use of Cyclic Peptide Libraries to Identify Optimal Protease Substrates

A cyclic peptide library was incubated with the protease of interest under reaction conditions where cleavage proceeded to a final end-state (e.g., no further cyclic peptide molecules are capable of serving as substrate for the protease), and the progress of the reaction monitored over time. Based on this information, a time point was chosen that corresponded to cleavage of only 2-20% of the library mixture, effectively selecting for the best protease substrates under the limiting conditions. The protease reaction was stopped, and the resulting mixture was then subjected to standard amino acid sequencing. The uncleaved cyclic molecules, lacking a free amino-terminus, were unable to participate in the Edman degradation reaction used for sequencing, and consequently did not contribute to the sequencing result. Similarly, because the protease of interest acted catalytically, and was present at <1:100 molar ratio compared with the cyclic peptide library, it was not necessary to remove the protease from the cleaved cyclic peptide products prior to sequencing (although this can easily be performed, if necessary, using standard size-exclusion chromatography).

As shown in FIG. 4, 500 μgs of a cyclic peptide library (sequence MAXXXXYXXXXARRN, cyclized head-to tail via a M-N peptide bond) was reacted with 5 μgs of bovine chymotrypsin in phosphate-buffered saline solution at room temperature for up to 120 min. Aliquots were removed at progressive time points, and protease-mediated cleavage of the cyclic library monitored by the appearance of free amino terminii as measured by reaction with ninhydrin. This analysis revealed complete cleavage of the cyclic peptide library within 40 minutes, with a linear rate of cleavage during the first 10 minutes. To determine the optimal protease substrate sequence, a

TABLE III

| | R | R | N | D | C | E | Q | G | H | I |
|---|---|---|---|---|---|---|---|---|---|---|
| Cycle | ALA | ARG | ASN | ASP | CYS | GLU | GLM | GLY | HIS | ILE |
| 1 | 610.4 | 59.3 | 51.1 | 33.4 | 0.0 | 59.3 | 42.2 | 141.4 | 22.0 | 86.9 |
| 2 | 598.8 | 82.0 | 57.2 | 32.1 | 0.0 | 44.6 | 42.4 | 353.1 | 47.0 | 85.9 |
| 3 | 261.9 | 168.4 | 60.1 | 49.3 | 0.0 | 77.7 | 49.5 | 153.3 | 47.8 | 71.6 |
| 4 | 242.0 | 173.9 | 155.4 | 57.7 | 0.0 | 67.1 | 45.7 | 106.6 | 42.0 | 62.1 |
| 5 | 920.9 | 167.1 | 156.6 | 59.8 | 0.0 | 39.0 | 23.7 | 56.1 | 31.5 | 21.7 |
| 6 | 250.5 | 316.5 | 100.0 | 39.0 | 0.0 | 25.1 | 13.1 | 29.9 | 20.4 | 13.1 |
| 7 | 246.3 | 422.2 | 110.7 | 47.7 | 0.0 | 29.4 | 12.7 | 35.9 | 18.2 | 11.7 |
| 8 | 135.9 | 273.5 | 471.8 | 72.4 | 0.0 | 52.8 | 12.3 | 41.9 | 15.9 | 12.4 |
| 9 | 126.3 | 173.7 | 154.1 | 41.2 | 0.0 | 55.6 | 11.8 | 34.8 | 12.6 | 11.2 |
| 10 | 408.5 | 175.7 | 110.1 | 37.6 | 0.0 | 37.2 | 11.3 | 35.0 | 10.7 | 11.6 |
| 11 | 137.5 | 134.9 | 83.8 | 38.0 | 0.0 | 16.7 | 13.0 | 59.3 | 10.7 | 16.8 |
| 12 | 96.3 | 112.1 | 58.0 | 31.0 | 0.0 | 10.7 | 13.9 | 45.7 | 9.9 | 15.3 |
| 13 | 83.3 | 139.0 | 43.1 | 35.0 | 0.0 | 16.3 | 20.0 | 52.9 | 12.5 | 15.9 |
| 14 | 54.9 | 85.1 | 37.7 | 23.7 | 0.0 | 11.2 | 10.2 | 33.1 | 8.0 | 11.2 |
| 15 | 33.3 | 77.8 | 27.8 | 19.7 | 0.0 | 7.5 | 6.2 | 18.5 | 6.1 | 6.2 |
| | L | K | M | F | P | S | T | W | Y | V |
| Cycle | LEU | LYS | MET | PHE | PRO | SER | THR | TRP | TYR | UGL |
| 1 | 104.8 | 148.2 | 231.8 | 59.7 | 10.9 | 98.7 | 75.3 | 4.5 | 77.7 | 180.6 |
| 2 | 182.0 | 109.2 | 172.6 | 104.9 | 61.8 | 59.0 | 48.6 | 3.9 | 161.8 | 165.4 |
| 3 | 99.9 | 110.6 | 122.3 | 66.3 | 148.7 | 53.2 | 51.8 | 3.3 | 142.9 | 146.6 |
| 4 | 65.4 | 99.7 | 91.8 | 56.0 | 171.8 | 44.0 | 44.1 | 2.8 | 131.6 | 121.9 |
| 5 | 30.4 | 46.8 | 193.0 | 26.8 | 79.5 | 18.2 | 15.9 | 1.9 | 71.4 | 45.1 |
| 6 | 16.7 | 26.0 | 172.9 | 14.3 | 38.8 | 9.2 | 8.0 | 1.1 | 126.4 | 25.0 |
| 7 | 17.5 | 22.1 | 88.5 | 14.5 | 43.4 | 9.7 | 8.5 | 0.9 | 100.3 | 25.5 |
| 8 | 19.4 | 23.5 | 94.2 | 15.9 | 46.3 | 11.0 | 9.9 | 1.8 | 54.5 | 28.7 |
| 9 | 18.5 | 19.7 | 492.1 | 13.8 | 40.8 | 9.1 | 8.6 | 0.7 | 37.6 | 24.8 |
| 10 | 17.4 | 19.3 | 93.9 | 14.0 | 37.1 | 10.2 | 9.0 | 0.6 | 32.9 | 25.1 |
| 11 | 23.3 | 25.2 | 72.5 | 19.8 | 53.9 | 14.4 | 13.0 | 0.8 | 77.1 | 37.1 |
| 12 | 22.2 | 21.8 | 55.9 | 16.9 | 52.0 | 11.9 | 11.2 | 0.6 | 80.7 | 35.6 |
| 13 | 23.3 | 22.8 | 51.1 | 17.8 | 43.6 | 15.3 | 14.8 | 0.7 | 61.7 | 36.5 |
| 14 | 17.1 | 12.4 | 29.8 | 14.0 | 10.4 | 9.8 | 9.3 | 0.5 | 50.9 | 27.3 |
| 15 | 9.7 | 10.2 | 31.0 | 10.2 | 20.9 | 5.6 | 5.0 | 0.2 | 160.1 | 13.7 |

second reaction was then performed for 8 minutes, at which point approximately 20% of the cyclic peptide library would have been cleaved. The protease reaction was terminated by addition of 1 mM phenylmethyl sulfonyl fluoride with heating to 100° C., and the entire mixture subjected to automated peptide sequencing on an Applied Biosystems Model 477A sequencer.

The resulting sequences in Table III revealed a marked excess of Tyr in position 15, confirming that cleavage occurred following this aromatic residue, as expected from the well-known specificity of chymotrypsin. To determine the optimal substrate sequence, the relative abundance of amino acids in positions flanking the tyrosine residue (e.g., cycles 11-14 which correspond to residues N-terminal to the tyrosine cleavage site, and cycles 1-4 which correspond to residues C-terminal to the tyrosine cleavage site), were compared with the abundance of each amino acid in the starting cyclic library mixture, based on amino acid sequencing following CNBr cleavage As shown in FIGS. 3A, 3B and 3C, this revealed a preference for Arg, His and Pro in the Tyr−2 position, Pro or Asn in the Tyr−1 position, and Ala, Lys or Val in the Tyr+1 position (See also, Table II). These findings are in excellent agreement with the preferred substrate specificity for chymotrypsin based on studies using synthetic linear di-and tripeptide analogues (Toszer et al., Acta Biochim. Biophys. Hung. 21:3351-348 (1986); Schellenberger et al, Eur. J. Biochem. 199:623-636 (1991)) and confirm the applicability of this technique.

### EXAMPLE 4

Use of Cyclic Peptide Library to Determine Protein Tyrosine Kinase Specificity

A Cyclic Peptide Library oriented around a fixed tyrosine residue was reacted with the protein tyrosine kinase src, in the presence of unlabelled and trace amounts of ³²P-γ-ATP until-1% of the peptides were radiolabelled. Enzyme and ATP were removed by DEAE column chromatography, and unphosphorylated peptides separated from phosphorylated peptides using a ferric chloride column. The purified phosphorylated cyclic peptides were cleaved by CNBr, and then subjected to peptide sequencing using an Applied Biosystems Model 477A sequencer.

TABLE IV
Substrate Specificity for the Protein Tyrosine Kinase src Assayed using
the Cyclic Peptide Library as Substrate

| pY − 4 | pY − 3 | pY − 2 | pY − 1 | pTyr | pY + 1 | pY + 2 | pY + 3 | pY + 4 |
|---|---|---|---|---|---|---|---|---|
| X | T(1.6) | T(3.6) | E(1.4) | | G(1.5) | E(1.6) | E(1.7) | X |
| | | | G(1.4) | | T(1.4) | G(1.6) | G(1.4) | |
| | | | | | E(1.3) | | S(1.4) | |

Following cyclic library phosphorylation, DEAE and FeCl₃ chromatography, and amino acid sequencing, the relative amino acid preferences were determined by normalizing the mole-percentage of each amino acid at each cycle from the Tyr-phosphorylated product to the mole-percentages present in the original un-phosphorylated cyclic library mixture. The results revealed a preference for Thr in position pTyr−2, as well as strong preferences for Glu and Gly in positions pY−1, pY+1, pY+2 and pY+3 as shown in Table IV. These sequences agreed with the predicted kinase substrate sequence (E/D-E/D-I/V/L-pTyr-G/E-E-F/I) determined using a linear peptide library technique (Songyang et al, Nature 373:536-539 (1995)).

### EXAMPLE 5

Combining Cyclic Peptide Library Screening of Proteases with Lipid Encapsulation

In this example, lipid encapsulation of proteases was combined with screening for optimal protease substrates using cyclic peptide libraries. By encapsulating the protease within a liposome prior to peptide library screening, only those cyclic peptides which are permeable to lipid bilayers (and therefore good drug candidates and candidate lead compounds) can enter the liposome and be cleaved by the protease.

A protease of interest is first encapsulated in liposomes using standard and previously published techniques (see e.g., G. Gregoriadis (1976) Methods Enzymol. 44:218-227). Following this, the liposome encapsulated protease is then incubated with the cyclic peptide library under conditions where 10-20% of the maximal cleavage (end state) occurs, as described in Example 3. The final mixture (cleaved and uncleaved peptides) is then immediately subjected to standard amino acid sequencing. Since the Edman reaction used in sequencing can only be performed when peptide molecules have a free amino-terminus (e.g., linear peptides), all of the uncleaved cyclic molecules are “invisible” and the resulting sequenced mixture uniformly identified only the optimal cyclic substrates for the protease.

Consequently, by combining the cyclic library screening with the lipid encapsulation of the enzymes, the search for cyclic-peptide-based anti-protease drug candidates is converted into a one-step screen that incorporates both optimal substrate selection and membrane permeability. Although described herein with regard to proteases, this approach of lipid encapsulation preceding cyclic library screening can also be used to identify the optimal cyclic peptide substrates of other enzymes that act on peptide substrates, including, but not limited to, kinases, phosphatases, methyltransferases, ubiquitinating enzymes and peptidyl-transferases.

### EXAMPLE 6

Use of Cyclic Peptide Libraries to Screen for Epitopes for Monoclonal Antibodies

Epitopes for antibodies (e.g., monoclonal antibodies) can be identified using an approach that takes advantage of linear peptide libraries to identify antibody epitopes comprising linear arrangements of particular amino acids and cyclic peptide libraries to identify antibody epitopes that comprise complex molecular surfaces. The screening procedure involves preparing immobilized monoclonal antibodies, incubating the immobilized antibodies with linear or cyclic peptide libraries, rapid removal of unbound library components, elution of the specific bound library peptides, followed by amino acid sequencing. During the initial screening process, if one component of the presumed epitope is known (e.g., phosphotyrosine or phosphothreonine), then screening is performed using libraries fixed around this invariant residue. If the epitope is completely unknown, then the screening is performed using a panel of 20 oriented libraries, each with a different amino acid as the fixed residue. (This approach both selects for key residues in the epitope and also provides confirmation once the final epitope sequence is deduced.) Once the epitope motif is established, a search of a protein sequence database (e.g., Genbank) can be performed to identify proteins that contain the epitope motif, thereby allowing for the identification of putative proteins that might cross-react with that monoclonal antibody. Applications of this approach include identifying epitopes for diagnostic and therapeutic monoclonal antibodies, and identifying the molecular targets of auto-antibodies in patients with autoimmune diseases.

### EXAMPLE 7

Generation of Diagnostic and Therapeutic Monoclonal Antibodies Using Cyclic Peptide Library Immunogens

The example describes an approach that can be used to generate a large panel of monoclonal antibodies that can then be rapidly examined for function, without having to establish the target protein of interest prior to antibody generation. Following identification of functional clones, epitope mapping and characterization of the molecular target is conducted as described in Example 6. This approach involves immunizing an animal with a library of peptides, followed by preparation of a panel of monoclonal antibodies from the immunized animal and selection of those monoclonal antibodies that have a desired functional property.

As an example, to generate therapeutic antibodies against signalling systems that mediate their effects using phosphoserine proteins, a phosphoserine library is coupled to keyhole limpet hemocyanin (KLH) and the library immunogen is used to inoculate mice. Using a library of ˜10⁷ peptides, perhaps ˜10³ will generate an antibody response. Splenocytes from immunized mice are then fused to hybridoma cells to produce monoclonal antibodies using standard techniques. These antibodies are then screened for the ability to interfere with the signalling reaction of interest (e.g., functional clones are identified) and the relevant clones are selected and expanded. Following selection, the epitopes of the selected antibodies can be mapped, and the native molecular targets characterized, using the approaches described in Example 6.

### EXAMPLE 8

Tag Termination Technique (TTT) for Duplex Screening of Linear and Cyclic Libraries to Identify Optimal Chain Length or Ring Size

In the tag termination technique (TTT), a portion of the growing peptide chains of the library is removed from the synthesizer at different chain lengths. Each portion is “coded” by introduction of a series of unique chemical tags whose cleavage can be performed prior to opening the ring or disrupting the linear peptide bond(s). Thus, a library is created that contains library members of different chain lengths (for linear peptides) ring sizes (for cyclic peptides), each being coded with a different tag. The tagged library is used to screen a binding protein of interest and library members are selected. Following screening, the selected library members are decoded by tag removal and analyzed as to their size to thereby identify the optimal size library for the binding protein of interest. The optimal sized library is then further screened using the standard library approach to determine the optimal binding motif of the binding protein of interest.

For example, one type of tag would involve the addition, to various portions of the growing peptide chains during synthesis, of a glutamate or aspartate residue containing alkyl-or aliphatic alcohol-thioester tags of varying chain length of the aliphatic chain of the tag is varied as the length of the library chains increases during synthesis. Following synthesis, the double degenerate library (length and sequence) is used to screen for target binding. The bound library peptides are eluted, the thioester of the tag is cleaved and the alkyl chain is separated from the peptides by reverse phase-HPLC or gas chromatography/mass spectroscopy. The peak in alkyl chain length identifies the optimum length or ring size for the library for the particular binding protein being analyzed. This optimal length library is then cleaved to remove the alkyl thioester and the library, free from the tag, is then used to screen for binding using the standard peptide library approach to identify the optimal substrate binding motif for the binding protein of interest.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method for determining an amino acid sequence motif for an interaction site of a binding compound, comprising:
contacting an oriented degenerate cyclic peptide library (ODCPL) with a binding compound under conditions which allow for interaction between the binding compound and the ODCPL, wherein the ODCPL comprises library members having an identifiable amino acid residue at a fixed non-degenerate position;
allowing the binding compound to interact with the ODCPL such that a complex is formed between the binding compound and a subpopulation of library members capable of interacting with the binding compound;
separating the subpopulation of library members capable of interacting with the binding compound from library members that are incapable of interacting with the binding compound;
linearizing the subpopulation of library members capable of interacting with the binding compound to form a subpopulation of linearized library members;
determining the amino acid sequence of the subpopulation of linearized library members; and
determining an amino acid sequence motif for an interaction site of the binding compound, based upon relative abundance of different amino acid residues at each degenerate position within the linearized library members.

2. The method of claim 1, wherein the ODCPL is a soluble synthetic peptide library.

3. The method of claim 1, wherein the ODCPL comprises cyclic peptides comprising a formula: [chemical structure]
wherein Zₐₐ is a non-degenerate natural or unnatural α-amino acid, Xₐₐ is any natural or unnatural α-amino acid, R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive.

4. The method of claim 3, wherein R—R′ is methionine-alanine.

5. The method of claim 4, wherein library members are linearized by cleaving R—R′ with cyanogen bromide.

6. The method of claim 1, wherein the binding compound is a kinase.

7. The method of claim 6, wherein the ODCPL comprises cyclic peptides comprising a formula: 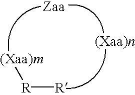
wherein Zₐₐ is a non-degenerate phosphorylatable amino acid selected from the group consisting of serine, threonine and tyrosine, Xₐₐ is any natural or unnatural α-amino acid, R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive.

8. The method of claim 7, wherein Zₐₐ is the only phosphorylatable amino acid within the cyclic peptides.

9. The method of claim 6, wherein the protein kinase is a protein-serine/threonine specific kinase and the ODCPL comprises cyclic peptides comprising a formula: 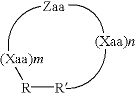
wherein Zₐₐ is a non-degenerate phosphorylatable amino acid selected from the group consisting of serine and threonine, Xₐₐ is any natural or unnatural α-amino acid, R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive.

10. The method of claim 6, wherein the protein kinase is a protein-tyrosine specific kinase and the ODCPL comprises cyclic peptides comprising a formula: 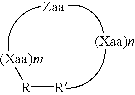
wherein Zₐₐ is tyrosine, Xₐₐ is any natural or unnatural α-amino acid , R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive.

11. The method of claim 6, wherein the kinase phosphorylates the subpopulation of library members capable of interacting with the kinase and then the subpopulation of phosphorylated library members is separated from nonphosphorylated library members.

12. The method of claim 11, wherein the subpopulation of phosphorylated library members is separated from nonphosphorylated library members by binding the subpopulation of phosphorylated library members to a ferric column or to an anti-phosphotyrosine antibody column.

13. The method of claim 1, wherein the binding compound is a phosphatase.

14. The method of claim 13, wherein the ODCPL comprises cyclic peptides comprising a formula: 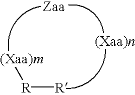
wherein Zₐₐ is a non-degenerate phosphorylated amino acid selected from the group consisting of phosphoserine, phosphothreonine and phosphotyrosine, Xₐₐ is any natural or unnatural α-amino acid, R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive.

15. The method of claim 14, wherein Z aa is the only phosphorylated amino acid within the cyclic peptides.

16. The method of claim 14, wherein the phosphatase dephosphorylates the subpopulation of library members capable of interacting with the phosphatase and then the subpopulation of dephosphorylated library members is separated from phosphorylated library members.

17. The method of claim 16, wherein the subpopulation of dephosphorylated library members is separated from phosphorylated library members by binding the phosphorylated library members to a ferric column or to an anti-phosphotyrosine.

18. The method of claim 1, wherein the binding compound comprises an SH2 domain.

19. The method of claim 18, wherein the ODCPL comprises cyclic peptides comprising a formula: 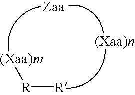
wherein Zₐₐ is a non-degenerate phosphotyrosine residue, Xₐₐ is any natural or unnatural α-amino acid, R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive.

20. The method of claim 18, wherein the binding compound comprising an SH2 domain is immobilized on a solid support, the ODCPL is passed over the solid support to allow the binding compound to interact with a subpopulation of library members capable of interacting with the SH2 domain, and library members incapable of interacting with the SH2 domain are washed away to thereby separate the subpopulation of library members capable of interacting with the SH2 domain from library members incapable of interacting with the SH2 domain.

21. The method of claim 1, wherein the binding compound comprises an SH3 domain.

22. The method of claim 21, wherein the ODCPL comprises cyclic peptides comprising a formula: 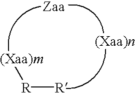
wherein Zₐₐ is a non-degenerate proline, Xₐₐ is any natural or unnatural α-amino acid, R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive.

23. The method of claim 21, wherein the binding compound comprising an SH3 domain is immobilized on a solid support, the ODCPL is passed over the solid support to allow the binding compound to interact with a subpopulation of library members capable of interacting with the SH3 domain, and library members incapable of interacting with the SH3 domain are washed away to thereby separate the subpopulation of library members capable of interacting with the SH3 domain from library members incapable of interacting with the SH3 domain.

24. The method of claim 1, wherein the binding compound is a protease and the subpopulation of library members capable of interacting with the protease are linearized by allowing the protease to cleave the library members.

25. The method of claim 1, wherein the binding compound is an antibody, or antigen-binding fragment thereof.

26. The method of claim 1, wherein the binding compound comprises a domain selected from the group consisting of WW domains, PTB domains, PDZ domains, LIM domains, pleckstrin homology domains and zinc finger domains.

27. The method of claim 1, wherein the binding compound is selected from the group consisting of extracellular growth factors, growth factor receptors, adhesion molecules, intercellular signaling molecules, lipid phosphatases, 7-transmembrane receptor proteins, proteases, ion channels, methyltransferases, ubiquitinating enzymes and peptidyl-transferases.

28. The method of claim 1, wherein the binding compound is encapsulated in a liposome prior to contacting the binding compound with the ODCPL.

29. An oriented degenerate cyclic peptide library (ODCPL), which comprises cyclic peptides comprising a formula: 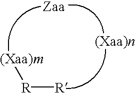
wherein Zₐₐ is a non-degenerate natural or unnatural α-amino acid, Xₐₐ is any natural or unnatural α-amino acid, R—R′ is a dipeptide specifically cleavable under cleavage conditions to allow for linearization of the peptide, and n and m are each independently selected from 0-10 inclusive, with the proviso that if n is 0, m is selected from 1-10 inclusive and if m is 0, n is selected from 1-10 inclusive.

30. The ODCPL of claim 29, wherein R—R′ is alanine-methionine.

31. The ODCPL of claim 29, wherein Zₐₐ is a non-degenerate phosphorylatable amino acid selected from the group consisting of serine, threonine and tyrosine.

32. The ODCPL of claim 29, wherein Zₐₐ is a non-degenerate phosphorylated amino acid selected from the group consisting of phosphoserine, phosphothreonine and phosphotyrosine.

33. The ODCPL of claim 32, wherein Zₐₐ is phosphotyrosine.

34. The ODCPL of claim 29, wherein Zₐₐ is proline.

35. The ODCPL of claim 29, wherein Zₐₐ is a nonnatural α-amino acid.

36. The ODCPL of claim 29, wherein Zₐₐ is a hydrophobic natural or nonnatural α-amino acid.

37. The ODCPL of claim 29, wherein Zₐₐ is a hydrophilic natural or nonnatural α-amino acid.

38. A method for purifying cyclic peptides from linear peptides comprising:
providing a mixture of cyclic peptides and linear peptides;
contacting the mixture with a blocking agent that reacts with the free amino termini of the linear peptides to form amino-protected linear peptides;
contacting the mixture with a binding agent that is capable of interacting with the amino-protected linear peptides but incapable of interacting with the cyclic peptides; and
separating the amino-protected linear peptides from the cyclic peptides to thereby purify the cyclic peptides.

39. The method of claim 38, wherein the blocking agent is biotin.

40. The method of claim 39, wherein the binding agent is avidin or streptavidin.

41. A method for determining an amino acid sequence motif for an interaction site of a protease, comprising:
contacting an oriented degenerate cyclic peptide library (ODCPL) with a protease under conditions which allow for interaction between the protease and the ODCPL, wherein the ODCPL comprises library members having an identifiable amino acid residue at a fixed non-degenerate position;
allowing the protease to interact with the ODCPL such that a complex is formed between the protease and a subpopulation of library members capable of interacting with the protease;
linearizing the subpopulation of library members capable of interacting with the protease to form a subpopulation of linearized library members;
determining the amino acid sequence of the subpopulation of linearized library members; and
determining an amino acid sequence motif for an interaction site of the protease, based upon relative abundance of different amino acid residues at each degenerate position within the linearized library members.

42. The method of claim 41, wherein said protease is chymotrypsin.

43. The method of claim 41, wherein said protease is from the serine protease family, cysteine protease family, metalloproteinase family, aminopeptidase family or carboxypeptidase family.
